(19) United States
(12) Patent Application Publication
(10) Pub. No.: US 2014/0255356 A1
(43) Pub. Date: Sep. 11, 2014

(54) ISOLATION OF STROMAL VASCULAR FRACTION FROM VASCULAR TISSUES

(71) Applicant: Steven VICTOR, New York, NY (US)

(72) Inventor: Steven VICTOR, New York, NY (US)

(21) Appl. No.: 14/199,121

(22) Filed: Mar. 6, 2014

Related U.S. Application Data
(60) Provisional application No. 61/773,482, filed on Mar. 6, 2013.
(60) Provisional application No. 61/793,934, filed on Mar. 15, 2013.

(51) Int. Cl. A61K 35/12; A61N 7/00

(52) U.S. Cl. CPC A61K 35/12; A61N 7/00; A61N 2007/0043
USPC 424/93.7; 435/173.9; 435/378; 435/379; 601/2

(57) ABSTRACT

Methods of isolating stromal vascular fractions for use in human subjects from a tissue comprising a blood vessel using mechanical, optionally ultrasonic cavitation, enzymatic, and/or chemical methods are provided. These methods yield an increased number of the cells which constitute the stromal vascular fractions (about 10-fold greater) than methods which use collagenase to isolate these cells. The stromal vascular fraction may be isolated from any tissue that comprises blood vessels, for example, muscle and adipose tissue. Additionally, the tissue may be isolated obtained from nonliving sources and animals (e.g., postmortem).

### FIELD OF THE INVENTION

The present invention relates to the isolation of a stromal vascular fraction a vascularized tissue obtained from living and non-living sources (e.g., post-mortem) using mechanical (e.g., ultrasonic cavitation), enzymatic, and/or chemical treatment, wherein the tissue cells and blood vessels are lysed thereby releasing intact stromal vascular fraction cells while substantially maintaining the viability of the stromal vascular fraction.

### BACKGROUND OF THE INVENTION

Within the last 10 years, adipose tissues have attracted attention as a resource of multipotent stem cells. See Zuk, et al. (2002) Mol Biol Cell. 13: 4279-4295. Adipose tissue comprises two fractions—the adipocyte fraction and the stromal-vascular fraction (SVF). Adipose tissue may be found in the visceral adipose tissue (VAT) and subcutaneous adipose tissue deposits (SAT). See Peinado, et al. (2010) Proteomics 10(18): 3356-66; Novakofski & Hu (1987) J Anim Sci 65: 12-24. The stromal vascular fraction (SVF) comprises preadipocytes, mesenchymal stem cells (MSC), endothelial progenitor cells, blood endothelial cells, fibroblasts, pericytes, T regulatory cells, and macrophages. Koh, et al. (2011) Arterioscler Thromb Vasc Biol. 31(5): 1141-50. The mesenchymanl stem cells (MSC) can be differentiated into a variety of cell lineages including adipogenic, chondrogenic, myogenic, and osteogeneic lineages. Rodriguez, et al. (2012) International Archives of Medicine 5: 1-9; Zuk, et al. (2001) Tissue Eng. 7: 211-228; Hicok, et al. (2004) Tissue Eng. 10: 371-380; Erickson, et al. (2002) Biochem Biophys Res Commun. 290: 763-769; Cousin, et al. (2003) Biochem Biophys Res Commun. 301: 1016-1022; Safford, et al. (2002) Biochem Biophys Res Commun. 294: 371-379; Miranville, et al. (2004) Circulation. 110: 349-355; Planat-Benard, et al. (2004) Circ Res. 94: 223-229; and Planat-Benard, et al. (2004) Circulation. 109: 656-663. The stromal vascular fraction, including the mesenchymal stem cells contained therein, are useful in research and therapeutics.

A common source of adipose tissue, including the stromal vascular fraction, is “lipoaspirate,” the adipose tissue harvested from patients undergoing liposuction. The lipoasperiate comprises adipocytes, fat, connective tissue, blood vessels, and stromal vascular fraction. The adipose tissue is usually separated from non-adipose tissue using a tissue collection container that utilizes decantation, sedimentation, and/or centrifugation techniques to separate the materials. The adipose tissue is then disaggregated using methods such as mechanical force (mincing or shear forces), enzymatic digestion with single or combinatorial protelolytic enzymes, such as collagenase, trypsin, lipase, liberase H1, pepsin, or a combination of mechanical and enzymatic methods. U.S. Pat. No. 7,429,488, For example, a lipoaspirate may be obtained. The fatty portion diluted with PBS and centrifuged to exclude all hematopoietic cells. The sample is then incubated with collagenase followed by centrifugation and filtration to isolate a stromal vascular fraction. Godthardt, et al. (2008) MACS Miltenyi Biotec Information Pamphlet. Other methods involve centrifugation and culturing the lipoaspirate. See Francis, et al. (2010) Organogenesis 6(1): 11-14. U.S. Patent Application Publication No. 2006/0051865 describes the use of ultrasonic methods to release adult stem cells from adipose tissue. U.S. Patent Application Publication 2007/0148766 also discloses the isolation of stem cells from liposuction-derived aspirates.

However, there exists a need in the art for new methods of isolating the stromal vascular fraction cells and new sources of stromal vascular fraction cells to meet the increasing demand for research and therapy.

### SUMMARY OF THE INVENTION

The invention provides for a method for isolating a stromal vascular fraction from a tissue comprising subjecting said tissue to mechanical, chemical, and/or enzymatic treatment to release the stromal vascular fraction.

In one embodiment, the method may comprise subjecting said tissue to ultrasonic cavitation, wherein the cells in the tissue and blood vessels in the tissue are lysed, thereby dissociating or releasing substantial numbers of intact stromal vascular fraction cells from the lysed blood vessels contained in the ultrasonicated tissue while substantially maintaining the viability of the cells constituting the stromal vascular fraction. In one embodiment, the method may comprise subjecting a tissue to ultrasonic cavitation comprising bringing said tissue into contact with a powerful ultrasonic cup horn, ultrasonic probe (rod), ultrasonic flow cell, or ultrasonic microtiter plate.

In one embodiment, the method may be performed in situ in a patient. In another embodiment, the method may further comprise removing the lysed tissue comprising the lysed blood vessels and the stromal vascular fraction, optionally adipose tissue. In another embodiment, the method may further comprise identifying and targeting the tissue using ultrasound. In another embodiment, the ultrasonic cavitation may be applied directly externally to the tissue, optionally applied to the patient's skin and the blood vessels are lysed to release the stromal vascular fraction. In another embodiment, the ultrasonic cavitation rod may be inserted through a puncture or incision in the skin into the patient's tissue and lyse the blood vessels to release stromal vascular fraction. In another embodiment, the tissue may be located using ultrasound device and then removed using indirect cavitation device and subject to ultrasonic cavitation to release the stromal vascular fraction. In another embodiment, the tissue may be, optionally located using ultrasound device, removed subject to ultrasonic cavitation by an ultrasonic flow cell device, optionally, an Ultrasonic Mini Flow Cell, to release the stromal vascular fraction, optionally, wherein the stromal vascular fraction is further isolated.

In one embodiment, the method may comprise providing about 40-60 mL of adipose tissue obtained from a nonliving animal; treating said adipose tissue with ultrasonic cavitation using an about 13-14 mm probe for about 10 minutes at about 24 kHz, wherein the adipose cells and blood vessels in the adipose tissue are lysed, thereby dissociating or releasing substantial numbers of intact stromal vascular fraction cells from the lysed blood vessels contained in the ultrasonicated adipose tissue while substantially maintaining the viability of the cells constituting the stromal vascular fraction. In another embodiment, the ultrasonic cavitation may be effected for about 5 minutes, paused, and then continued for another 5 minutes for a total of 10 minutes. In another embodiment, the probe may be placed towards the bottom of the tissue sample for a first 5 minute period, paused, and then the probe is moved upwards to about half-way in the tissue sample and continued for the second 5 minute period. In another embodiment, the ultrasonic cavitation may be at a frequency of about 20-30 kHz, optionally about 20, 21, 22, 23, 24, 24, or 25 kHz, or optionally about 20-23 kHz or 23-25 kHz.

In one embodiment, the ultrasonic cavitation may be performed using an ultrasonic probe of about 10-15 mm, about 13 or 14 mm probe, optionally about 14 mm. In another embodiment, the probe may be 14 mm. In another embodiment, the ultrasonic cavitation device may have a 200 W-500 W generator, optionally a 200 W generator.

In another embodiment, the method may comprise providing a tissue obtained from an animal; mixing said a tissue with tumescent fluid; and homogenizing said tissue with beads for about 30 seconds to 6 minutes, wherein the tissue cells and blood vessels in the tissue are lysed, thereby dissociating or releasing substantial numbers of intact stromal vascular fraction cells from the lysed blood vessels contained in the tissue while substantially maintaining the viability of the cells constituting the stromal vascular fraction. In another embodiment, the method may further comprise isolating the stromal vascular fraction (SVF), optionally removing the beads. In another embodiment, the adipose tissue may be comminuted prior to homogenization with beads. In another embodiment, the beads may be stainless steel, zirconium oxide, tungsten carbide, ceramic, zirconium silicate, or glass beads, optionally zirconium oxide beads. In another embodiment, the beads may be about 0.01-2 mm beads, optionally 2 mm beads. In another embodiment, the beads may be added at a bead-to-sample ratio of about 1:1-1:4, optionally 1:4 (25% beads by volume).

In one embodiment, the sample may comprise about 7 mL of tissue, optionally adipose tissue. In another embodiment, the sample may be mixed with about 3 mL of tumescent fluid. In another embodiment, the method may comprise providing about 7 mL of tissue obtained from an animal; mixing said tissue with about 3 mL of tumescent fluid; homogenizing said tissue with about 5 mL of 2 mm zirconium oxide beads for about 3 minutes at a bead-to-sample ratio of 1:3, wherein the tissue cells and blood vessels in the tissue are lysed, thereby dissociating or releasing substantial numbers of intact stromal vascular fraction cells from the lysed blood vessels contained in the tissue while substantially maintaining the viability of the cells constituting the stromal vascular fraction; and isolating the stromal vascular fraction (SVF) cells, optionally removing the beads. In another embodiment, the sample may be homogenized with beads for about 3 minutes.

In one embodiment, the tissue may be mechanically treated using a homogenizer, optionally a rotor stator homogenizer; a dounce; mortar and pestle; tissue mill, mixer-mill, or bead-beater assembly; blender; spin column homogenizer; or a sonicator. In another embodiment, the tissue may be mechanically comminuted, optionally by grinding, dicing, slicing, chopping up, comminuting, grinding with mortar and pestal, granulating, pressing, cubing, mincing, milling, grating, grading, crushing, rolling, shearing, dividing, or hewing.

In one embodiment, after the mechanical comminuting, the tissue may be further treated with enzymes, chemicals, or ultrasonic cavitation. In another embodiment, the tissue may be treated using enzymes, optionally catabolic enzymes, optionally enzymes that degrade proteins, carbohydrates, lipids, DNA, RNA, and other cellular and non-cellular molecules. In another embodiment, the enzyme may be a protease, collagenase, elastase, hyaluronidase, trypsin, chymotrypsin, papain, proteinase K, lipase, DNase, optionally exonucleases and endonucleases, optionally DNase I, DNase II, or Shrimp arctic DNase, RNases, amylases, and/or cellulases. In another embodiment, the enzyme may be a catabolic enzyme, optionally a protease, optionally Proteinase K, Subtilisin, selected from the group consisting of B. amyloliquefaciens Subtilisin also known as Subtilisin BPN, Subtilisin novo, BAS, Neutrase, bacterial protease Novo, furilysin, Nagarse, subtilopeptidase B, subtilopeptidase C, or Subtilisin B; B. licheniformis Subtilisin, Subtilisin A, thiosubtilisin; a cysteine protease, optionally papain, or a keratinase. In another embodiment, the method may comprise the use of a combination of at least two, three, four, five, six, seven, eight, nine, ten, eleven, twelve, thirteen, fourteen, or more catabolic enzymes, optionally admixed together with or without a nuclease inhibitor. In another embodiment, the enzyme may be used in a concentration range of between about 0.001 mg/ml to about 50 mg/ml, optionally about 0.01 mg/ml to about 5 mg/ml, or between about 0.2 mg/ml to about 1.0 mg/ml.

In one embodiment, the amount of tissue may be about 50 mL. In another embodiment, the sample may comprise about 40, 45, 50, 55, or 60 cc of tissue.

In one embodiment, the method may further comprise isolating the stromal vascular fraction (SVF).

In one embodiment, the method does not include the addition of an endopeptidase, optionally collagenase. In another embodiment, the method may comprise the addition of endopeptidase, optionally collagenase.

In one embodiment, the animal may be a mammal, optionally a human.

In one embodiment, the tissue may be obtained from the stromal or mesenchymal compartment of a human cadaver, tissue bank, organ donation, solid fat obtained from a human cadaver, or a liposuction derived aspirate. In another embodiment, the animal may be at least about 1, 2, 3, 4, 5, 6, 7, 8, 9, 10, 11, 12, 13, 14, 15, 16, 17, 18, 19, 20, 21, 22, 23, or 24 hours post-mortem. In another embodiment, the animal may be at least about 1, 2, 3, 4, 5, 6, 7, 8, 9, or days post-mortem.

In one embodiment, the tissue may be comprised in phosphate buffered saline, normal saline, or another biologically acceptable liquid.

In one embodiment, the tissue may comprise blood vessels. In another embodiment, the tissue may be selected from the group consisting of connective tissue, muscle tissue, adipose tissue, nervous tissue, and epithelial tissue. In another embodiment, the connective tissue may be selected from the group consisting of blood, bone, and extracellular matrix. In another embodiment, the nervous tissue may be selected from the group consisting of neural tissue selected from the group consisting of central nervous system may comprise the brain and spinal cord and peripheral nervous system may comprise cranial nerves and spinal nerves may comprise motor neurons. In another embodiment, the muscle tissue may be selected from the group consisting of skeletal (striated) muscle, cardiac muscle, and smooth muscle. In another embodiment, the epithelial tissue may be selected from the group consisting of squamous epithelium, cuboidal epithelium, columnar epithelium, glandular epithelium, and ciliated epithelium.

In one embodiment, the method may further comprise allowing the treated tissue, optionally adipose tissue, to settle or may be centrifuged, optionally for about 3 minutes at 500 RCF (relative centrifugal force), resulting in the fat rising to the top of the sample.

In one embodiment, the stromal vascular fraction may comprise mesenchymal stem cells, hematopoietic cells, hematopoietic stem cells, platelets, Kupffer cells, osteoclasts, megakaryocytes, granulocytes, NK cells, endothelial precursor or progenitor cells, CD34+ cells or mesenchymal stem cells, CD29+ cells, CD166+ cells, Thy-1+ stem cells, CD90+ stem cells, CD44+ cells, monocytes, leukocytes, lymphocytes, B cells, T cells, NK cells, macrophages, neutrophil leukocytes, neutrophils, and neutrophil granulocytes.

In one embodiment, after the mechanical, enzymatic, and/or chemical treatment, the sample may be assayed, optionally by flow cytometry, for the presence of adipose-derived stem cells including CD34 and/or Thy-1 or CD90 expressing stem cells. In another embodiment, after the mechanical, enzymatic, and/or chemical treatment, the sample may be fractionated using fluorescence activated call sorting (FACS) based on cell surface antigens which are specific to stem cells, optionally mesenchymal stem cells, endothelial precursor cells, or hematopoietic stem cells. In another embodiment, the method may further comprise isolating the stromal vascular fraction and cryopreserving said stromal vascular fraction. In another embodiment, the method may result in a yield of at least about 1×10⁶ to 1×10⁷ stromal vascular cells per mL of adipose tissue. In another embodiment, at least about 80%, 85%, 90%, 91%, 92%, 93%, 94%, 95%, 96%, 97%, 98%, 99%, or 100% of said stromal vascular fraction cells isolated may be viable. In another embodiment, the method may further comprise isolating stem cells, optionally mesenchymal stem cells, endothelial precursor cells, or hematopoietic stem cells, from said stromal vascular fraction.

In one embodiment, the isolated stromal vascular fraction derived from adipose tissue may not comprise any exogenous collagenase. In another embodiment, the stromal vascular fraction may comprise hematopoietic elements. In another embodiment, the stromal vascular fraction may comprise extracellular matrix (ECM) materials. In another embodiment, white blood cells may have been removed from the stromal vascular fraction. In another embodiment, the stromal vascular fraction may be essentially free of white blood cells. In another embodiment, the T cells may be depleted from the stromal vascular cells. In yet another embodiment, the stromal vascular cells may be essentially free of T cells.

In one embodiment, a use of the stromal vascular fraction may be used in the manufacture of cosmetic surgery products, optionally dermal fillers.

In one embodiment, a method for cosmetic surgery may comprise administering a stromal vascular fraction.

In one embodiment, a composition for cosmetic surgery may comprise an effective amount of stromal vascular fraction cells. In another embodiment, the stromal vascular fraction may be used in a cosmetic surgery application, to promote wound healing, are used in a tissue filler or in association with breast augmentation, breast reconstruction, tissue engineering, or burn treatment.

In one embodiment, a use of the stromal vascular fraction in the manufacture of a medicament for the treatment of a disease.

In one embodiment, a method of treating a disease may comprise administering a stromal vascular fraction.

In one embodiment, a composition for treating a disease may comprise an effective amount of stromal vascular fraction cells.

In one embodiment, a use of the stromal vascular fraction in the manufacture of medicament for allogeneic transplantation to treat a disease.

In one embodiment, a method for treating a disease may comprise administering an allogeneic transplant may comprise stromal vascular cells.

In one embodiment, a composition for allogeneic transplantation may comprise an effective amount of stromal vascular fraction cells.

In one embodiment, a method for treating a disease may comprise obtaining stromal vascular cells from a patient and administering the stromal vascular cells to the same patient to treat said disease.

In one embodiment, a method for xenotransplantation may comprise obtaining stromal vascular cells from an animal and transplanting them into a human patient, optionally to treat a disease.

In one embodiment, a method for allogeneic transplantation may comprise transplanting may comprise stromal vascular cells. In another embodiment, the patent receiving the transplant may be different from the patient from whom the stromal vascular cells were obtained.

In one embodiment, a pharmaceutical composition for the treatment of a disease may comprise stromal vascular cells.

In one embodiment, a pharmaceutical composition for the treatment of a disease may comprise stromal vascular cells. In another embodiment, the composition may further comprise tissue fillers. In another embodiment, the use, method, or composition may comprise treatment alone or in combination with tissue fillers.

In one embodiment, a method for treating a disease may comprise administering a composition comprising stromal vascular cells into the bone marrow of a patient in need thereof.

In one embodiment, a method for treating a disease may comprise isolating stromal vascular cells from a vascular tissue and administering it to a patent in need thereof.

In one embodiment, a method for treating a disease may comprise matching a stromal vascular fraction donor with a recipient by blood type and/or HLA, isolating stromal vascular cells by ultrasonic cavitation from a vascular tissue from said donor and administering said stromal vascular cells to said recipient.

In one embodiment, the composition comprising stromal vascular cells may be administered to iliac crest.

In one embodiment, the disease may be leukemia, aplastic anemia, lymphoma, optionally Hodgkin's disease, non-Hodgkin's lymphoma, multiple myeloma, immune deficiency disorders, a solid tumor, optionally breast or ovarian cancer, or sickle cell disease.

In another embodiment, the T cells are depleted from the stromal vascular cells.

In another embodiment, the disease may be gum recession, loss of bone, including the jaw, amyotrophic lateral sclerosis (ALS), arthritis, optionally rheumatoid arthritis, autism, diabetes, optionally Type I diabetes, bone fractures, chronic obstructive pulmonary disease (COPD), dermal treatment for burns and non-healing wounds, enterocutaneous fistula (HULPUTC), gingival gum regeneration, hair loss (in both men and women), gum recession, ischemic heart failure, microvascular protection treatment in a myocardial infarction, migraine, multiple sclerosis, orthopedic problems, osteoarthritis, plantar fascitis, recto-vaginal fistula, rheumatoid arthritis, rotator cuff injuries, sports medicine injuries, optionally tears and sprains of the ligaments and tendons, tennis elbow, tinnitus, ulcers, and non-healing wounds.

In one embodiment, the method of providing a stromal vascular fraction composition to a clinical site may comprise (a) thawing vials of cryopreserved stromal vascular fraction cells, (b) resuspending the stromal vascular fraction cells in media, (c) centrifuging the stromal vascular fraction cells, (d) resuspending the stromal vascular fraction cells in media, (e) aliqouting the stromal vascular fraction cells into vials, and (f) transferring to the clinical site. In another embodiment, the resuspension and centrifugation steps may be repeated at least 1, 2, 3, 4, or 5 times. In another embodiment, the stromal vascular fraction product may be transported to the clinical site within at least about 1, 2, 3, 4, 5, 6, 7, 8, 9, or 10 hours of completion of step (e). In another embodiment, the vials may be labeled.

In one embodiment, the method for a providing stromal vascular fraction cell preparation for sale may comprise (a) producing stromal vascular fraction cells and (b) preparing said stromal vascular fraction cell preparations for transfer to a customer. In another embodiment, the method may comprise cryopreserving the stromal vascular fraction cells. In another embodiment, the method may comprise offering said stromal vascular fraction cell preparations for sale. In another embodiment, the said method may comprise advertising the stromal vascular fraction cell preparations.

In one embodiment, a SVF composition for use in the manufacture of cosmetic surgery products, optionally may comprise dermal fillers.

In one embodiment, a method for cosmetic surgery may comprise administering a SVF composition.

In one embodiment, a composition for cosmetic surgery may comprise an effective amount of the SVF composition.

In one embodiment, a SVF composition for use in the manufacture of cosmetic surgery products, optionally may comprise dermal fillers, optionally for the reduction of a skin defect.

In one embodiment, a method for reducing a skin defect may comprise administering a SVF composition.

In one embodiment, a composition for reduction of a skin defect may comprise an effective amount of a SVF composition.

In one embodiment, a SVF composition for use in the manufacture of cosmetic surgery products, optionally may comprise dermal fillers, for reducing wrinkles, improving rosacea, increasing skin thickness, increasing skin tone, improving skin texture, or tightening skin.

In one embodiment, a method for reducing wrinkles, improving rosacea, increasing skin thickness, increasing skin tone, improving skin texture, or tightening skin may comprise administering a SVF composition.

In one embodiment, a composition for reducing wrinkles, improving rosacea, increasing skin thickness, increasing skin tone, improve skin texture, and tighten skin may comprise an effective amount of the SVF composition. In another embodiment, the use, method, or composition may be for the reduction of a skin defect. In another embodiment, the skin defect may be a dynamic wrinkle, a fine wrinkles or a static wrinkle. In another embodiment, the dynamic wrinkle may be a forehead crease, a brow burrow or an eye line (crow's feet). In a further embodiment, the static wrinkle may be a skin fold wrinkle resulting from sagging skin. In a further embodiment, the skin defect may be a medical condition selected from the group consisting of an acne scar, optionally a “rolling” scar, a “boxcar” scar or an “ice pick” scar, a surgical scar, trauma scar, a large pore and a soft tissue contour defect.

In another embodiment, the wrinkle or scar may be the result of loss of collagen and hyaluronic acid in the skin during the aging process. In a further embodiment, the wrinkle or scar may be the result of premature aging, optionally premature aging caused by overexposure to sunlight, overexposure to environmental pollutants, smoking tobacco products, exposure to cigarette smoke, poor nutrition, and skin disorders.

In one embodiment, a method for treating rosacea, psoriasis, acne, eczema, and atopic dermatitis may comprise administering a SVF composition, optionally the SVF composition may be applied topically.

In one embodiment, a cosmetic method for treating wrinkles, tone, text, large pores, dullness, or loose skin, may comprise administering an effective amount of SVF optionally the SVF composition may be applied topically.

In one embodiment, a method for augmenting soft tissue to provide relief of a skin defect may comprise administering an effective amount of SVF.

In one embodiment, the method for augmenting soft tissue to provide reduction of a skin defect may comprise topically applying to the skin defect a composition may comprise SVF. In another embodiment, the skin defect may be the result of loss of collagen and hyaluronic acid in the skin during the aging process. In another embodiment, the skin defect may be the result of premature aging, said premature aging caused by overexposure to sunlight, overexposure to environmental pollutants, smoking tobacco products, exposure to cigarette smoke, poor nutrition, and skin disorders.

In one embodiment, a method for augmenting soft tissue to provide reduction of a skin defect, optionally wherein the skin defect may be a dynamic wrinkle, a fine wrinkles or a static wrinkle, may comprise administering an effective amount of SVF. In another embodiment, the dynamic wrinkle may be a forehead crease, a brow burrow or an eye line (crow's feet). In another embodiment, the static wrinkle may be a skin fold wrinkle resulting from sagging skin. In a further embodiment, the skin defect may be a medical condition selected from the group consisting of an acne scar, for example, a “rolling” scar, a “boxcar” scar or an “ice pick” scar, a surgical scar, trauma scar, a large pore and a soft tissue contour defect. In a further embodiment, the medical condition may be a deformity that requires re-contouring, such as a small tissue defect, optionally after animal bite(s)) or a deformity related to trauma wherein the deformity is cosmetically unappealing. In another embodiment, the augmentation may be done after plastic surgery to achieve symmetry or a desired result.

In one embodiment, a method of augmenting soft tissue to provide long-term reduction of a skin defect may comprise administering an effective amount of SVF.

In another embodiment, the long-term reduction of a skin defect may be of a duration of at least one year, one year to about five years, five years to about ten years, or ten years or longer.

In one embodiment, a method of recovering a stromal vascular fraction from tissue, optionally connective, muscle, nervous, adipose, and/or epithelial tissue, may comprise providing about 40-60 mL, optionally 50 mL, of tissue obtained from a nonliving animal; treating said tissue with ultrasonic cavitation using an about 13-14 mm probe for about 10 minutes at about 24 kHz, wherein the adipose cells and blood vessels in the adipose tissue are lysed, thereby dissociating or releasing substantial numbers of intact stromal vascular fraction cells from the lysed blood vessels contained in the ultrasonicated tissue while substantially maintaining the viability of the cells constituting the stromal vascular fraction. In another embodiment, the tissue may be adipose tissue.

In one embodiment, a method for isolating a stromal vascular fraction for a tissue may comprise: (a) identifying the tissue using ultrasound; (b) subjecting said tissue to ultrasonic cavitation comprising bringing said tissue into contact with a powerful ultrasonic cup horn, ultrasonic probe (rod), ultrasonic flow cell, or ultrasonic microtiter plate, wherein said ultrasonic cavitation is applied directly externally to the tissue, optionally applied to the patient's skin, and wherein the blood vessels are lysed, dissociating or releasing substantial numbers of intact stromal vascular fraction cells from the lysed blood vessels contained in the ultrasonicated tissue while substantially maintaining the viability of the cells constituting the stromal vascular fraction, and (c) removing the lysed tissue comprising the lysed blood vessels and the stromal vascular fraction. In another embodiment, said method may be performed in situ in a patient. In another embodiment, said tissue may be adipose tissue.

In one embodiment, a method for isolating a stromal vascular fraction from a tissue may comprise: (a) identifying the tissue using ultrasound; (b) subjecting said tissue to ultrasonic cavitation comprising inserting a cavitation rod through a puncture or incision in the skin into the patient's tissue and lyse the blood vessels to release stromal vascular fraction while substantially maintaining the viability of the cells constituting the stromal vascular fraction, and (c) removing the lysed tissue comprising the lysed blood vessels and the stromal vascular fraction.

In one embodiment, a method for isolating a stromal vascular fraction from a tissue may comprise (a) subjecting said tissue to ultrasonic cavitation comprising inserting a cannulae through a puncture or incision in the skin into the patient's tissue, and (b) extracting the tissue comprising blood vessels and the stromal vascular fraction. In another embodiment, the tissue may be subject to indirect ultrasonic cavitation to release the stromal vascular fraction. In another embodiment, the ultrasonic cavitation may be by an ultrasonic flow cell device, optionally an Ultrasonic Mini Flow Cell, powerful ultrasonic cup horn, ultrasonic probe (rod), ultrasonic flow cell, or ultrasonic microtiter plate.

In another embodiment, the method may further comprise isolating the stromal vascular fraction (SVF). In a further embodiment, the stromal vascular fraction cells may be cultured and/or expanded. In a further embodiment, mesenchymal stem cells may be isolated from the stromal vascular fraction. In a further embodiment, the mesenchymal stem cells may be cultured and/or expanded.

In another embodiment, the method may not include the addition of an endopeptidase, optionally collagenase. In another embodiment, the method may include the addition of an endopeptidase, optionally collagenase.

In another embodiment, the animal may be a mammal, optionally a human. In another embodiment, the tissue, optionally connective, muscle, nervous, adipose, and/or epithelial tissue, may be obtained from the stromal or mesenchymal compartment of a human cadaver, solid fat obtained from a human cadaver, or a liposuction derived aspirate. In another embodiment, the animal may be at least about 1, 2, 3, 4, 5, 6, 7, 8, 9, 10, 11, 12, 13, 14, 15, 16, 17, 18, 19, 20, 21, 22, 23, or 24 hours post-mortem. In a further embodiment, the animal may be at least about 1, 2, 3, 4, 5, 6, 7, 8, 9, or 10 days post-mortem. In another embodiment, the tissue, optionally connective, muscle, nervous, adipose, and/or epithelial tissue, may be obtained from a tissue bank, organ donation, or stored (e.g., frozen tissue).

In another embodiment, the tissue, optionally connective, muscle, nervous, adipose, and/or epithelial tissue, may be comprised in phosphate buffered saline (PBS), Krebs-Ringer-bicarbonate buffer, normal saline, RIPA lysis buffer, or another biologically acceptable buffer.

In another embodiment, the ultrasonic cavitation may be effected for about 5 minutes, paused, and then continued for another 5 minutes for a total of 10 minutes. In another embodiment, the probe may be placed towards the bottom of the tissue, optionally connective, muscle, nervous, adipose, and/or epithelial tissue, sample for a first 5 minute period, paused, and then the probe may be moved upwards to about half-way in the adipose tissue sample and continued for the second 5 minute period. In another embodiment, the ultrasonic cavitation devices may have a 200 W generator and a rod size of about 13-14 mm. In another embodiment, the ultrasonic cavitation device may have a 200 W-500 W generator or a 200 W generator.

In another embodiment, the adipose sample may comprise about 40, 45, 50, 55, or 60 mL of tissue, optionally connective, muscle, nervous, adipose, and/or epithelial tissue.

In another embodiment, the method may further comprise allowing the treated adipose tissue to settle or may be centrifuged, optionally for about 3 minutes at 500 RCF (relative centrifugal force), resulting in the fat rising to the top of the sample, forming a pellet may comprise the SVF cells and a supernatant comprises the fat.

In another embodiment, the stromal vascular fraction may comprise mesenchymal stem cells, hematopoietic cells, hematopoietic stem cells, platelets, Kupffer cells, osteoclasts, megakaryocytes, granulocytes, NK cells, endothelial precursor or progenitor cells, CD34+ cells or mesenchymal stem cells, CD29+ cells, CD166+ cells, Thy-1+ stem cells, CD90+ stem cells, CD44+ cells, monocytes, leukocytes, lymphocytes, B cells, T cells, NK cells, macrophages, neutrophil leukocytes, neutrophils, and neutrophil granulocytes.

In another embodiment, after ultrasonic cavitation, the sample may be assayed, optionally by flow cytometry, for the presence of adipose-derived stem cells including CD34 and/or Thy-1 or CD90 expressing stem cells.

In another embodiment, after ultrasonic cavitation, the sample may be fractionated using fluorescence activated call sorting (FACS) based on cell surface antigens which are specific to adipose-derived stem cells.

In another embodiment, the method may further comprise isolating the stromal vascular fraction and cryopreserving said stromal vascular fraction.

In another embodiment, the method may result in a yield of at least about 1×106 to 1×107 stromal vascular cells per mL of tissue, optionally connective, muscle, nervous, adipose, and/or epithelial tissue.

In a further embodiment, the method may comprise aliqouting the treated adipose tissue sample with equal amounts of 0.9% Sodium Chloride. In another embodiment, the adipose tissue/0.9% Sodium Chloride mixture may be centrifuged.

In another embodiment, the adipose tissue may be treated with ultrasonic cavitation and then centrifuged to remove the fat.

In another embodiment, at least about 80%, 85%, 90%, 91%, 92%, 93%, 94%, 95%, 96%, 97%, 98%, 99%, or 100% of said stromal vascular fraction cells isolated are viable.

In a further embodiment, the stromal vascular fraction may be located in blood vessels contained in or proximate to a tissue, optionally connective, muscle, nervous, adipose, and/or epithelial tissue.

In one embodiment, an isolated stromal vascular fraction derived from tissue, optionally connective, muscle, nervous, adipose, and/or epithelial tissue, by the method described herein and does not comprise any exogenous collagenase.

In one embodiment, a method of recovering a stromal vascular fraction tissue, optionally connective, muscle, nervous, adipose, and/or epithelial tissue, may comprise providing about 50-60 cc of tissue obtained from a nonliving animal in a 60 cc tube size, about 28 mm in diameter and about 110 mm in length; treating said tissue with ultrasonic cavitation using an about 13-14 mm diameter probe, optionally a 14 mm probe, for about 10 minutes at about 24 kHz, for two 5 minute periods, optionally consecutive, where the first 5 minute period of ultrasonic cavitation may be at the lower portion of the sample and the second 5 minute period of ultrasonic cavitation may be about half-way in the sample, wherein the cells and blood vessels in the tissue are lysed, thereby dissociating or releasing substantial numbers of intact stromal vascular fraction cells from the lysed blood vessels contained in the ultrasonicated adipose tissue while substantially maintaining the viability of the cells constituting the stromal vascular fraction.

In another embodiment, the stromal vascular fraction may comprise stem cells that express at least one protein selected from the group consisting of CD13, CD29, CD34, CD36, CD44, CD49d, CD54, CD58, CD71, CD73, Thy-1, CD90, CD105, CD106, CD151, and SH3. In another embodiment, the stromal vascular fraction may comprise stem cells that express at least one protein selected from the group consisting of CD31, CD45, CD117, and CD146. In another embodiment, the stromal vascular fraction may comprise stem cells that do not express CD56. In another embodiment, the stromal vascular fraction may comprise stem cells that do not express at least one protein selected from the group consisting of CD3, CD4, CD14, CD15, CD16, CD19, CD33, CD38, CD56, CD61, CD62e, CD62p, CD69, CD104, CD135, and CD144. In another embodiment, the stromal vascular fraction may comprise stem cells that express CD49d but do not express CD56. In a further embodiment, the stromal vascular fraction may comprise mesenchymal stem cells that have the following immunophenotype CD73+, CD90+, CD105+, CD11b/14−, CD19/CD73b−, CD34−, CD45−, and HLA-DR−.

The invention provides a method of recovering a stromal vascular fraction from tissue, optionally connective, muscle, nervous, adipose, and/or epithelial tissue, may comprise providing adipose tissue obtained from an animal; mixing said tissue with tumescent fluid; homogenizing said tissue with beads for about 30 seconds to 6 minutes, wherein the adipose cells and blood vessels in the tissue are lysed, thereby dissociating or releasing substantial numbers of intact stromal vascular fraction cells from the lysed blood vessels contained in the ultrasonicated tissue while substantially maintaining the viability of the cells constituting the stromal vascular fraction.

In one embodiment, the method may further comprise isolating the stromal vascular fraction (SVF), optionally removing the beads.

In one embodiment, the animal may be a mammal, optionally a human. In a further embodiment, the tissue may be connective, muscle, nervous, adipose, epithelial tissue, or a mixture thereof. In another embodiment, the adipose tissue may be obtained from the stromal or mesenchymal compartment of a human cadaver, tissue bank, organ donation, solid fat obtained from a human cadaver, or a liposuction derived aspirate. In another embodiment, the tissue, optionally connective, muscle, nervous, adipose, and/or epithelial tissue, may be comminuted prior to homogenization with beads.

In one embodiment, the animal may be at least about 1, 2, 3, 4, 5, 6, 7, 8, 9, 10, 11, 12, 13, 14, 15, 16, 17, 18, 19, 20, 21, 22, 23, or 24 hours post-mortem. In another embodiment, the animal may be at least about 1, 2, 3, 4, 5, 6, 7, 8, 9, or 10 days post-mortem.

In one embodiment, the beads may be stainless steel, zirconium oxide, tungsten carbide, ceramic, zirconium silicate, or glass beads, optionally zirconium oxide beads. In another embodiment, the beads may be about 0.01-2 mm beads, optionally 2 mm beads.

In one embodiment, the adipose sample may comprise about 7 mL of adipose tissue. In another embodiment, the adipose sample may be mixed with about 3 mL of tumescent fluid. In another embodiment, the adipose sample may be homogenized with beads for about 3 minutes.

In one embodiment, the beads may be added at a bead-to-sample ratio of about 1:1-1:4, optionally 1:4 (25% beads by volume).

In one embodiment, the method of recovering a stromal vascular fraction from adipose tissue may comprise providing about 7 mL of adipose tissue obtained from an animal, optionally lipoaspirate; mixing said adipose tissue with about 3 mL of tumescent fluid; homogenizing said adipose tissue with about 5 mL of 2 mm zirconium oxide beads for about 3 minutes at a bead-to-sample ratio of about 1:3 or about 1:4, wherein the adipose cells and blood vessels in the adipose tissue may be lysed, thereby dissociating or releasing substantial numbers of intact stromal vascular fraction cells from the lysed blood vessels contained in the ultrasonicated adipose tissue while substantially maintaining the viability of the cells constituting the stromal vascular fraction; and isolating the stromal vascular fraction (SVF) cells, optionally removing the beads.

In one embodiment, a method for cosmetic surgery may comprise administering a stromal vascular fraction obtained by the method described herein. In a further embodiment, a use of the stromal vascular fraction obtained by a method described herein may be used in the manufacture of cosmetic products. In another embodiment, a method for tissue transplantation, optionally allogeneic or autologous, may comprise administering a stromal vascular fraction obtained by a method described herein. In a further embodiment, the use of the stromal vascular fraction obtained by a method described herein may be used in tissue transplantation, optionally allogeneic or autologous transplantation. In another embodiment, the stromal vascular fraction may be used in a cosmetic surgery application, to promote wound healing, may be used in a tissue filler or in association with breast augmentation or reconstruction, tissue engineering, or burn treatment.

In one embodiment, a method of recovering a stromal vascular fraction from adipose tissue may comprise providing about 7 mL of adipose tissue, optionally obtained from a nonliving animal; treating said adipose tissue homogenization for about 30-60 seconds with 2 mm zirconium oxide beads, wherein the adipose cells and blood vessels in the adipose tissue may be lysed, thereby dissociating or releasing substantial numbers of intact stromal vascular fraction cells from the lysed blood vessels contained in the adipose tissue while substantially maintaining the viability of the cells constituting the stromal vascular fraction.

In another embodiment, the animal may be a mammal, optionally a human. In another embodiment, the tissue, optionally connective, muscle, nervous, adipose, and/or epithelial tissue, may be obtained from the stromal or mesenchymal compartment of a human cadaver, solid fat obtained from a human cadaver, or a liposuction derived aspirate. In another embodiment, the animal may be at least about 1, 2, 3, 4, 5, 6, 7, 8, 9, 10, 11, 12, 13, 14, 15, 16, 17, 18, 19, 20, 21, 22, 23, or 24 hours post-mortem. In a further embodiment, the animal may be at least about 1, 2, 3, 4, 5, 6, 7, 8, 9, or 10 days post-mortem. In another embodiment, the tissue, optionally connective, muscle, nervous, adipose, and/or epithelial tissue, may be obtained from a tissue bank, organ donation, or stored (e.g., frozen tissue).

In one embodiment, the adipose sample may comprise about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 cc of adipose tissue. In a further embodiment, the tissue, optionally connective, muscle, nervous, adipose, and/or epithelial tissue, may comprise about 7, 8, 9, or 10 cc of tissue, optionally connective, muscle, nervous, adipose, and/or epithelial tissue. In another embodiment, the adipose sample may comprise up to about 60 cc of adipose tissue or about 20-60 cc tissue, optionally connective, muscle, nervous, adipose, and/or epithelial tissue.

In one embodiment, a method of recovering a stromal vascular fraction adipose tissue may comprise providing about 5-10 mL of adipose tissue, optionally obtained from a nonliving animal, in a 50 cc size tube; treating said adipose tissue with bead homogenization for 30-60 seconds with 0.1 mm beads, or from about 30 seconds to 6 minutes, wherein the adipose cells and blood vessels in the adipose tissue may be lysed, thereby dissociating or releasing substantial numbers of intact stromal vascular fraction cells from the lysed blood vessels contained in the homogenized adipose tissue while substantially maintaining the viability of the cells constituting the stromal vascular fraction.

In one embodiment, a method for cosmetic surgery may comprise administering a stromal vascular fraction obtained by the method described herein. In one embodiment, the use of the stromal vascular fraction obtained by the method described herein may be used in the manufacture of cosmetic products. In another embodiment, the stromal vascular fraction may be used in a cosmetic surgery application, to promote wound healing, are used in a tissue filler or in association with breast augmentation or reconstruction, tissue engineering, or burn treatment.

In one embodiment, a method of treating a disease may comprise administering a stromal vascular fraction obtained by the method described herein. In one embodiment, the use of the stromal vascular fraction obtained by the method described herein in the manufacture of a medicament for the treatment of a disease. In another embodiment, the method and use may include treatment alone or in combination with tissue fillers. The stromal vascular fraction cells (SVF), optionally mesenchymal stem cells derived therefrom, in uses, methods, and compositions for treating amyotrophic lateral sclerosis (ALS), arthritis, optionally rheumatoid arthritis, autism, diabetes, optionally Type I diabetes, bone fractures, chronic obstructive pulmonary disease (COPD), dermal treatment for burns and non-healing wounds, enterocutaneous fistula (HULPUTC), gingival gum regeneration, hair loss (in both men and women), gum recession, ischemic heart failure, microvascular protection treatment in a myocardial infarction, migraine, multiple sclerosis, orthopedic problems, osteoarthritis, plantar fascitis, recto-vaginal fistula, rheumatoid arthritis, rotator cuff injuries, sports medicine injuries, optionally tears and sprains of the ligaments and tendons, tennis elbow, tinnitus, ulcers, or non-healing wounds.

In one embodiment, the stromal vascular fraction cells, optionally mesenchymal stem cells derived therefrom, may be used in compositions for the treatment of loss of bone, optionally the jaw, amyotrophic lateral sclerosis (ALS), arthritis, optionally rheumatoid arthritis, autism, diabetes, optionally Type I diabetes, bone fractures, chronic obstructive pulmonary disease (COPD), dermal treatment for burns and non-healing wounds, enterocutaneous fistula (HULPUTC), gingival gum regeneration, hair loss (in both men and women), gum recession, ischemic heart failure, microvascular protection treatment in a myocardial infarction, migraine, multiple sclerosis, orthopedic problems, osteoarthritis, plantar fascitis, recto-vaginal fistula, rheumatoid arthritis, rotator cuff injuries, sports medicine injuries, optionally tears and sprains of the ligaments and tendons, tennis elbow, tinnitus, ulcers, or wounds may comprise an effective amount of stromal vascular fraction cells, optionally mesenchymal stem cells derived therefrom.

In another embodiment, the stromal vascular fraction cells may be used in methods for the treatment of loss of bone, optionally the jaw, amyotrophic lateral sclerosis (ALS), arthritis, optionally rheumatoid arthritis, autism, diabetes, optionally Type I diabetes, bone fractures, chronic obstructive pulmonary disease (COPD), dermal treatment for burns and non-healing wounds, enterocutaneous fistula (HULPUTC), gingival gum regeneration, hair loss (in both men and women), gum recession, ischemic heart failure, microvascular protection treatment in a myocardial infarction, migraine, multiple sclerosis, orthopedic problems, osteoarthritis, plantar fascitis, recto-vaginal fistula, rheumatoid arthritis, rotator cuff injuries, sports medicine injuries, optionally tears and sprains of the ligaments and tendons, tennis elbow, tinnitus, ulcers, or wounds may comprise administering an effective amount of stromal vascular fraction cells, optionally mesenchymal stem cells derived therefrom to a subject in need thereof.

In one embodiment, the stromal vascular fraction obtained by the method described herein may be used in the manufacture of cosmetic surgery products, optionally dermal fillers.

In one embodiment, a method for cosmetic surgery may comprise administering a stromal vascular fraction obtained by the method described herein.

In one embodiment, a composition for cosmetic surgery may comprise an effective amount of stromal vascular fraction cells obtained by the method described herein.

In another embodiment, the stromal vascular fraction may be used in a cosmetic surgery application, to promote wound healing, may be used in a tissue filler or in association with breast augmentation, breast reconstruction, tissue engineering, or burn treatment.

In another embodiment, the use of the stromal vascular fraction obtained by the methods described herein may be in the manufacture of a medicament for the treatment of a disease.

In another embodiment, the method of treating a disease may comprise administering a stromal vascular fraction obtained by the methods described herein.

In another embodiment, the composition for treating a disease may comprise an effective amount of stromal vascular fraction cells obtained by the methods described herein.

In another embodiment, the use of the stromal vascular fraction obtained by the method described herein may be in the manufacture of medicament for allogeneic transplantation to treat a disease.

In another embodiment, the method for treating a disease may comprise administering an allogeneic transplant may comprise stromal vascular cells obtained by the methods described herein. In a further embodiment, a method for allogeneic transplantation may comprise transplanting may comprise stromal vascular cells obtained by the methods described herein into a patient. In another embodiment, the patent receiving the transplant may be different from the patient from whom the stromal vascular cells were obtained.

In another embodiment, the composition for allogeneic transplantation may comprise an effective amount of stromal vascular fraction cells obtained by the methods described herein.

In another embodiment, the method for treating a disease may comprise obtaining stromal vascular cells by the methods described herein from a patient and administering the stromal vascular cells to the same patient to treat said disease.

In another embodiment, the method for xenotransplantation may comprise obtaining stromal vascular cells from an animal and transplanting them into a human patient, optionally to treat a disease.

In another embodiment, the pharmaceutical composition for the treatment of a disease may comprise stromal vascular cells. In another embodiment, the pharmaceutical composition for the treatment of a disease may comprise stromal vascular cells by the methods described herein. In another embodiment, the composition may further comprise tissue fillers. In another embodiment, the use, method, or composition may comprise treatment alone or in combination with tissue fillers.

In another embodiment, the disease may be gum recession, loss of bone, including the jaw, amyotrophic lateral sclerosis (ALS), arthritis, optionally rheumatoid arthritis, autism, diabetes, optionally Type I diabetes, bone fractures, chronic obstructive pulmonary disease (COPD), dermal treatment for burns and non-healing wounds, enterocutaneous fistula (HULPUTC), gingival gum regeneration, hair loss (in both men and women), gum recession, ischemic heart failure, microvascular protection treatment in a myocardial infarction, migraine, multiple sclerosis, orthopedic problems, osteoarthritis, plantar fascitis, recto-vaginal fistula, rheumatoid arthritis, rotator cuff injuries, sports medicine injuries, optionally tears and sprains of the ligaments and tendons, tennis elbow, tinnitus, ulcers, and non-healing wounds.

In one embodiment, a method for isolating a stromal vascular fraction from a tissue may comprise (a) subjecting said tissue to ultrasonic cavitation comprising bringing said tissue into contact with a powerful ultrasonic cup horn, ultrasonic probe (rod), ultrasonic flow cell, or ultrasonic microtiter plate, optionally wherein said method is performed in situ in a patient, wherein the cells in the tissue and blood vessels in the tissue are lysed, thereby dissociating or releasing substantial numbers of intact stromal vascular fraction cells from the lysed blood vessels contained in the ultrasonicated tissue while substantially maintaining the viability of the cells constituting the stromal vascular fraction, and (b) removing the lysed tissue comprising the lysed blood vessels and the stromal vascular fraction. In another embodiment, the tissue may be adipose tissue.

In one embodiment, a cosmetic composition may comprise an effective amount of an allogeneic stromal vascular fraction. In another embodiment, the composition may be in a form selected from the group consisting of a balm, solution, suspension, emulsion, ointment, foam, paste, gel, cream, lotion, powder, salve, soap, surfactant-containing cleansing, oil, serum, drops, liposomes, nanoparticles, nanoboots, and spray. In another embodiment, the ccomposition may be formulated for topical administration.

In one embodiment, a cosmetic method for treating wrinkles, tone, text, large pores, dullness, or loose skin, may comprise administering an effective amount of an allogeneic stromal vascular fraction composition, optionally the allogeneic SVF composition may be applied topically.

In one embodiment, the allogeneic hematopoietic stem cells may be used in lieu of bone marrow for therapeutic uses.

In another embodiment, the method may further comprises administering stem cells, optionally mesenchymal stem cells, endothelial precursor cells, or hematopoietic stem cells.

In one embodiment, the isolated stromal vascular fraction may not comprise any exogenous collagenase.

In one embodiment, the stromal vascular fraction may comprise hematopoietic elements.

In one embodiment, the stromal vascular fraction comprises extracellular matrix (ECM) materials.

In one embodiment, white blood cells may have been removed from the stromal vascular fraction. In another embodiment, stromal vascular fraction may be essentially free of white blood cells. In another embodiment, SVF may be isolated from an organ comprising blood vessels. In another embodiment, the organ may be heart, lung, liver, bladder, kidney, pancreas, or stomach.

In one embodiment, the composition may be in a form selected from the group consisting of a balm, solution, suspension, emulsion, ointment, foam, paste, gel, cream, lotion, powder, salve, soap, surfactant-containing cleansing, oil, serum, drops, liposomes, nanoparticles, nanoboots, and spray. In another embodiment, the composition is a cream, lotion, or solution.

In one embodiment, a method for formulating an autologous skin cream may comprise isolating SVF from a patient and compounding to form a skin cream comprising autologous SVF for said patient. In another embodiment, SVF composition may be administered topically.

### BRIEF DESCRIPTION OF THE FIGURES

FIG. 1 depicts a schematic of indirect cavitation device where the tissue sample (crude emulsion) enters at the first end and passes through a glass tube where a sonicator subjects the tissue sample to ultrasonic cavitation. Pressurized water runs in the opposite direction outside the glass tube. The tissue sample remains in a sealed, sterile environment and thus there is no contamination from handling.

FIG. 2 is a schematic of an indirect ultrasonic cavitation system. The tissue sample may be extracted in a syringe and placed in a syringe pump that forces the tissue same into the indirect cavitation device where the tissue sample is subjected to ultrasonic cavitation, the treated tissue sample may then be collected in a filter to allow the separation of debris from the stromal vascular fraction.

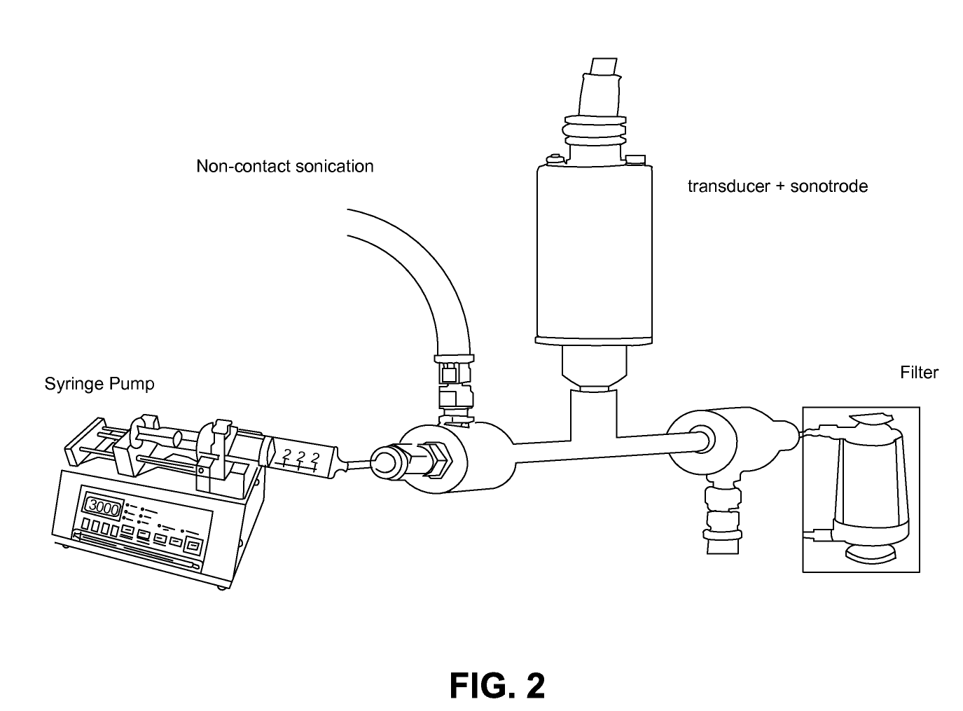

FIG. 3 is a schematic of a non-contact indirect ultrasonic cavitation device. The tissue may be removed by wand aspirator and fed into the indirect cavitation device where the tissue sample (crude emulsion) enters at the first end and passes through a glass tube where a sonicator subjects the tissue sample to ultrasonic cavitation. Pressurized water runs in the opposite direction outside the glass tube. The tissue sample remains in a sealed, sterile environment and thus there is no contamination from handling. Also depicted is a water return and filer. The water return pressurizes water and feeds the pressurized water intake after the water runs over the glass tube inside the indirect cavitation device, the pressurized water can return to be filtered, chilled, and returned to the indirect cavitation device. Also depicted is a filter system where the treated tissue sample may then be collected in a filter to allow the separation of debris from the stromal vascular fraction.

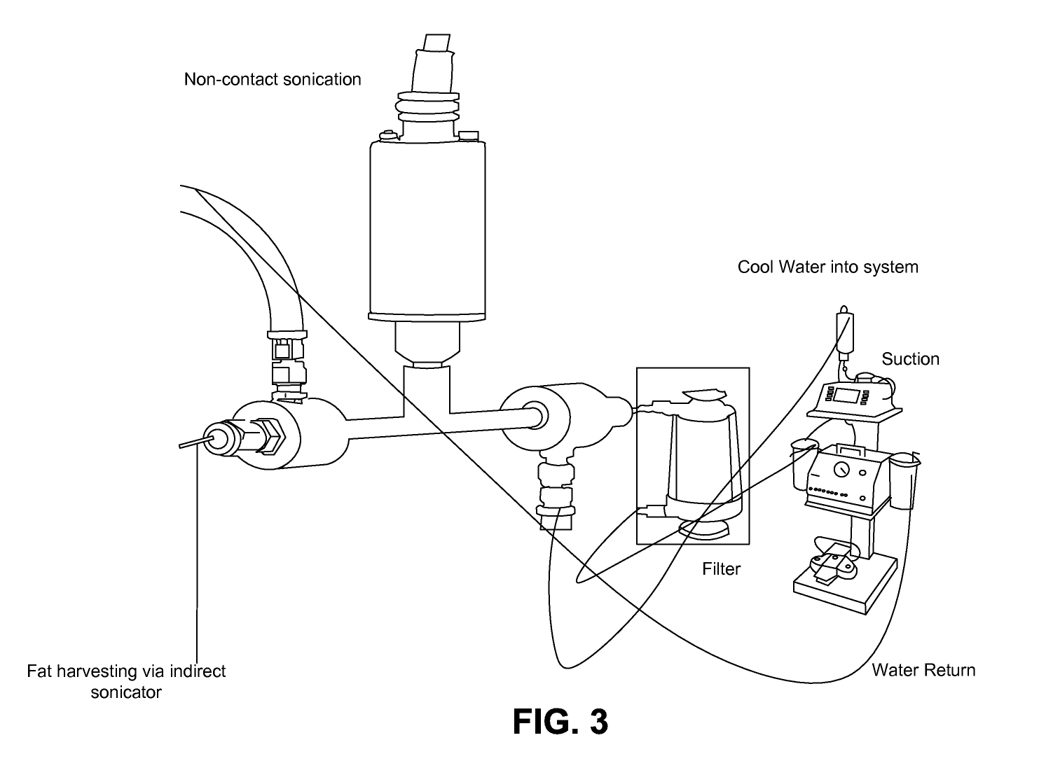

### DETAILED DESCRIPTION OF THE PREFERRED EMBODIMENTS

In order that the invention herein described may be fully understood, the following detailed description is set forth. Various embodiments of the invention are described in detail and may be further illustrated by the provided examples. Additional viable variations of the embodiments can easily be envisioned.

### DEFINITIONS

Unless defined otherwise, all technical and scientific terms used herein have the same meaning as those commonly understood by one of ordinary skill in the art to which this invention belongs.

As used in the description herein and throughout the claims that follow, the meaning of “a,” “an,” and “the” includes plural reference unless the context clearly dictates otherwise.

“About,” will be understood by persons of ordinary skill in the art and will vary to some extent based on the context in which it is used.

“Adipose tissue-derived cell,” as used herein, refers broadly to a cell that originates in adipose tissue, from the blood vessels contained therein. The initial cell population isolated from adipose tissue is a heterogeneous cell population including, but not limited to, stromal or mesenchymal vascular fraction (SVF).

“Adipose tissue,” as used herein, refers broadly to any fat tissue. The adipose tissue may be brown or white adipose tissue. The adipose may be mesenchymal or stromal. Preferably, the adipose tissue is subcutaneous white adipose tissue. The adipose tissue may be from any organism having fat tissue. Preferably the adipose tissue is mammalian, most preferably the adipose tissue is human. A convenient source of human adipose tissue is that derived from liposuction surgery or other surgery. Adipose tissue may be obtained from non-living donors, including animals, including mammals, post-mortem.

“Adipose-derived stem cell (ADSC or ASC),” as used herein, refers broadly to mesenchymal stem cells that originate from blood vessels found in adipose tissue which can serve as stem cell-like precursors to a variety of different cell types including but not limited to adipocytes, osteocytes, chondrocytes, muscle and neuronal/glial cell lineages. Adipose-derived stem cells make up a subset population derived from adipose tissue which can be separated from other components of the adipose tissue using standard culturing procedures or other methods disclosed herein. In addition, adipose-derived adult stem cells can be isolated from a mixture of cells using the cell surface markers disclosed herein. Also, adipose-derived stem cells are known as mesenchymal stem cells in the art.

“Adipose cell,” as used herein, refers broadly to any type of adipose tissue, including an undifferentiated adipose-derived adult stem cell and a differentiated adipose-derived adult stem cell.

“Allogeneic,” as used herein, refers broadly to any material derived from a different mammal of the same species.

“Allograft,” as used herein, refers broadly to a tissue graft from a donor genetically unrelated to the recipient.

“Allotransplantation,” as used herein, refers broadly to the transplantation of cells, tissues, or organs, to a recipient from a (genetically non-identical) donor from the same species. Allotransplants may be referred to an allograft, allogeneic transplant, or homograft in the art.

“Applicator,” as used herein, refers broadly to any device including, but not limited to, a hypodermic syringe, a pipette, for administering the compounds and compositions of the invention.

“Autograft,” as used herein, refers broadly to a tissue transplanted from one site to another on the same patient (e.g., removal of SVF cells and transplant to another site).

“Autologous,” as used herein, refers broadly to any material derived from the same individual to which it is later to be re-introduced.

“Central nervous system,” as used herein, refers broadly to include brain and/or the spinal cord of a mammal. The term may also include the eye and optic nerve in some instances.

“Cosmetically or aesthetically effective amount,” as used herein, refers broadly to a compound or cells is that amount of compound or cells which is sufficient to provide a cosmetically or aesthetically beneficial effect to the subject to which the compound or cells are administered such as skin rejuvenation, enhancement in plumpness or volume or appearance of treated tissue such as the cheeks, lips, buttocks, or breast tissue. Also, as used herein, a “cosmetically effective amount” is the amount of cells which is sufficient to provide a beneficial effect to the subject to which the cells are administered.

“Differentiated,” as used herein, refers broadly to a cell that has achieved a terminal state of maturation such that the cell has developed fully and demonstrates biological specialization and/or adaptation to a specific environment and/or function. Typically, a differentiated cell is characterized by expression of genes that encode differentiation-associated proteins in that cell. For example expression of GALC in a leukocyte is a typical example of a terminally differentiated leukocyte.

“Differentiation medium,” as used herein, refers broadly to a cell growth medium comprising an additive or a lack of an additive such that a stem cell, adipose tissue derived stromal cell, embryonic stem cell, ES-like cell, MSCs, neurosphere, NSC or other such progenitor cell, that is not fully differentiated when incubated in the medium, develops into a cell with some or all of the characteristics of a differentiated cell.

“Differentiating,” as used herein, refers broadly to a cell that is in the process of being differentiated.

“Differentiated adipose-derived adult stem cell” refers broadly to an adipose-derived adult stem cell isolated from any adipose tissue that has differentiated as defined herein.

“Undifferentiated adipose-derived adult stem cell,” as used herein, refers broadly to a cell isolated from adipose tissue and cultured to promote proliferation, but has no detectably expressed proteins or other phenotypic characteristics indicative of biological specialization and/or adaptation.

“Effective amount,” as used herein, refers broadly to a compound is that amount of compound or cells which is sufficient to provide a beneficial effect to the subject to which the compound is administered. Also, as used herein, an “effective amount” is the amount of cells which is sufficient to provide a beneficial effect to the subject to which the cells are administered.

“Endogenous,” as used herein, refers broadly to any material from or produced inside an organism, cell or system.

“Exogenous,” as used herein, refers broadly to any material introduced from or produced outside an organism, cell, or system. In particular exogenous may refer to a material that is not present in the treated adipose tissue.

“Isolated,” as used herein, refers broadly to material removed from its original environment in which it naturally occurs, and thus is altered by the hand of man from its natural environment. Isolated material may be, for example, exogenous nucleic acid included in a vector system, exogenous nucleic acid contained within a host cell, or any material which has been removed from its original environment and thus altered by the hand of man (e.g., “isolated cell”). For example, “isolated” or “purified,” as used herein, refers broadly to a protein, cell, DNA, antibody, RNA, or biologically active portion thereof, that is substantially free of cellular material or other contaminating proteins from the cell or tissue source from which the biological substance is derived, or substantially free from chemical precursors or other chemicals when chemically synthesized. For example, “isolated cell” refers broadly to a cell which has been separated from other components and/or cells which naturally accompany the isolated cell in a tissue or mammal.

“Isograft,” as used herein, refers broadly to a transplanted organ or tissue from a genetically identical donor (i.e., identical twin).

“Graft,” as used herein, refers broadly to a cell, tissue or organ that is implanted into an individual, typically to replace, correct or otherwise overcome a defect. A graft may further comprise a scaffold. The tissue or organ may consist of cells that originate from the same individual; this graft is referred to herein by the following interchangeable terms: “autograft”, “autologous transplant”, “autologous implant” and “autologous graft”. A graft comprising cells from a genetically different individual of the same species is referred to herein by the following nterchangeable terms: “allograft”, “allogeneic transplant”, “allogeneic implant” and “allogeneic graft”. A graft from an individual to his identical twin is referred to herein as an “isograft”, a “syngeneic transplant”, a “syngeneic implant” or a “syngeneic graft”. A “xenograft”, “xenogeneic transplant” or “xenogeneic implant” refers to a graft from one individual to another of a different species.

“Immunophenotype,” as used herein, refers broadly to cell is used herein to refer to the phenotype of a cell in terms of the surface protein profile of a cell.

“Late passaged adipose tissue-derived stromal cell,” as used herein, refers broadly to a cell exhibiting a less immunogenic characteristic when compared to an earlier passaged cell. The immunogenicity of an adipose tissue-derived stromal cell corresponds to the number of passages. Preferably, the cell has been passaged up to at least the second passage, more preferably, the cell has been passaged up to at least the third passage, and most preferably, the cell has been passaged up to at least the fourth passage.

“Mammal,” as used herein, refers broadly to any and all warm-blooded vertebrate animals of the class Mammalia, including humans, characterized by a covering of hair on the skin and, in the female, milk-producing mammary glands for nourishing the young. Examples of mammals include but are not limited to alpacas, armadillos, capybaras, cats, camels, chimpanzees, chinchillas, cattle, dogs, elephants, goats, gorillas, hamsters, horses, humans, lemurs, llamas, mice, non-human primates, pigs, rats, sheep, shrews, squirrels, tapirs, and voles. Mammals include but are not limited to bovine, canine, equine, feline, murine, ovine, porcine, primate, and rodent species. Mammal also includes any and all those listed on the Mammal Species of the World maintained by the National Museum of Natural History, Smithsonian Institution in Washington D.C.

“Mesenchymal stromal cells” or “Mesenchymal stem cells,” as used herein, refers broadly to cells derived from a stromal vascular fraction that have a multipotent differentiation potential (e.g., adipogenic, chondrogenic, and osteogenic) and may show CD73⁶⁺, CD90⁺, CD105⁺, CD11b/14⁻, CD19/CD73b⁻, CD34⁻, CD45⁻, and HLA-DR⁻. See, e.g., Gimble, et al. (2011) Stem Cells 29: 749-754 and Alexander (2012) Journal of Prolotherapy [online].

“Multipotential” or “multipotentiality,” as used herein, refers broadly to the capability of a stem cell to differentiate into more than one type of cell.

“Phenotypic characteristics,” as used herein, refers broadly to mean at least one of the following characteristics: morphological appearance, the expression of a specific protein, a staining pattern or the ability to be stained with a substance.

“Precursor cell,” “progenitor cell,” and “stem cell” are used interchangeably in the art and herein and refer broadly either to a pluripotent, or lineage-uncommitted, progenitor cell, which is potentially capable of an unlimited number of mitotic divisions to either renew itself or to produce progeny cells which will differentiate into the desired cell type. In contrast to pluripotent stem cells, lineage-committed progenitor cells are generally considered to be incapable of giving rise to numerous cell types that phenotypically differ from each other. Instead, progenitor cells give rise to one or possibly two lineage-committed cell types.

“Stromal vascular fraction,” as used herein, refers broadly to a cell fraction derived from blood vessels found in adipose tissue that comprises different cell types including by way of example mesenchymal stem cells, hematopoietic cells, hematopoietic stem cells, platelets, Kupffer cells, osteoclasts, megakaryocytes, granulocytes, NK cells, endothelial precursor or progenitor cells, CD34+ cells, CD29+ cells, CD166+ cells, Thy-1+ or CD90+ stem cells, CD44+ cells, immune cells such as monocytes, leukocytes, lymphocytes, B and T cells, NK cells, macrophages, neutrophil leukocytes, neutrophils, neutrophil granulocytes, and the like including immune and other cells that express one or more of the following markers: CD3, CD14 (macrophage marker), CD19, CD20 (B cell marker), CD29 (integrin unit), CD31 (endothelial, platelet, macrophage, Kupffer cell, dendritic cell, granulocyte, T/NK cells, lymphocytes, megakaryocytes, osteoclasts, neutrophils, et al.), CD44 (Hyaluronic acid receptor), CD45 (B and T cell marker), C56, CD73 (lymphocyte differentiation marker), CD105. Also, it includes cells expressing any of the markers or any combination thereof disclosed in this application.

“Treat,” as used herein, refers broadly to reduce the frequency of the disease or disorder reducing the frequency with which a symptom of the one or more symptoms disease or disorder is experienced by an animal.

“Vascularized” or “vascular,” as used herein, refers broadly to of, relating to, affecting, or consisting of a vessel or vessels, especially. those that carry blood. Vascularized refers to any tissue that comprises a blood vessel.

“Xenogeneic,” as used herein, refers broadly to any material derived from a mammal of a different species.

“Xenograft,” as used herein, refers broadly to a transplant from another species (e.g., transplant from a donor of one species to receipt of another different species).

### Tissue Sources

The inventor surprisingly discovered that the methods described herein may be used to isolate stromal vascular fraction (SVF) including stromal vascular fraction cells from a vascularized tissue source. This was unexpected because stromal vascular fraction has only been isolated from adipose tissue. The inventor surprisingly discovered that stromal vascular fraction cells can be isolated from any tissue source comprising blood vessels including but not limited to adipose tissue, optionally subcutaneous fat.

The inventors also surprisingly discovered that stromal vascular cells (SVF) may be isolated from blood vessels in any tissue. Thus, the inventors surprisingly discovered that stromal vascular fraction cells may be isolated from any tissue including but not limited to connective, muscle, nervous, adipose, and epithelial tissue. Stromal vascular fraction cells may be isolated from connective tissue including but not limited to blood, bone, and extracellular matrix. Further, stromal vascular fraction cells may be isolated from nervous tissue including but not limited to neural tissue including but not limited to central nervous system comprising the brain and spinal cord, or peripheral nervous system comprise cranial nerves and spinal nerves including motor neurons. Stromal vascular fraction cells may be isolated from muscle tissue including but not limited to skeletal (striated) muscle, cardiac muscle, or smooth muscle. Stromal vascular fraction cells may be isolated from adipose tissue. Stromal vascular fraction cells may be isolated from epithelial tissue including but not limited to squamous epithelium, cuboidal epithelium, columnar epithelium, glandular epithelium, and ciliated epithelium.

The adipose tissue treated by the claimed method described herein may be obtained from a variety of living and non-living sources including but not limited to animals (e.g., cows, chickens, sheep, goats, pigs) and humans (e.g., lipoaspirate, removed during surgery, or from cadavers).

To isolate the stromal vascular fraction, the tissue may be subject to mechanical, chemical, and/or enzymatic treatment to release the stromal vascular fraction.

### Enzymatic Methods for Treating Tissue

The tissue may be treated using enzymes, optionally catabolic enzymes including but not limited to enzymes that can degrade proteins, carbohydrates, lipids, DNA, RNA, and other cellular and non-cellular molecules. For example, proteases, collagenases, elastases, hyaluronidases, trypsins, chymotrypsins, papain, proteinase K, lipases, DNases (e.g., exonucleases and endonucleases, including but not limited to DNase I, DNase II, and Shrimp arctic DNase), RNases, amylases, and/or cellulases may be used to treat the tissue to release the stromal vascular fraction. A catabolic enzyme, optionally a protease may be used. The protease can be, for example, Proteinase K (a member of the broad family of Subtilisin-like enzymes.) The protease may also be Subtilisin including but not limited to B. amyloliquefaciens Subtilisin also known as Subtilisin BPN, Subtilisin novo, BAS, Neutrase, bacterial protease Novo, furilysin, Nagarse, subtilopeptidase B, subtilopeptidase C, or Subtilisin B; B. licheniformis Subtilisin also known as Subtilisin Carlsberg, Alcalase, Maxatase, Versazyme® Keratinase (strain PWD-1) from BioResource International, Inc., Subtilisin A, or thiosubtilisin; B. licheniformis engineered Subtilisin, Purafect®, Purafect Ox®, or Properase® from Genencor; B. lentus Subtilisin also known as Savinase, Everlase a protein-engineered variant of Savinase®, Esperase, Maxacal, protease PB92 (Bacillus sp.), Subtilisin 309, or Subtilisin BL; B. subtilis Subtilisin also known as Subtilisin E′ or Subtilisin 147. Any engineered Subtilisin (e.g., chemical or molecular engineered) or derivatives of the Subtilisins discussed herein can be used with the present invention. The protease can be a cysteine protease (e.g., papain), or a keratinase.

Also, the enzymatic approach may comprise the use of a combination of at least two, three, four, five, six, seven, eight, nine, ten, eleven, twelve, thirteen, fourteen, or more catabolic enzymes. The catabolic enzymes can be admixed together in any number of combinations. The catabolic enzymes can be admixed together with or without the nuclease inhibitor.

The concentration range of a given catabolic enzyme can be, for example, between about 0.001 mg/ml to about 50 mg/ml. The range can be between from about 0.01 mg/ml to about 5 mg/ml, or between about 0.2 mg/ml to about 1.0 mg/ml. The concentration can be about 0.4 mg/ml. Two or more catabolic enzymes may be used at the same or different amounts.

### Chemical Methods for Treating Tissue

The tissue may be treated using chemicals to hydrolyze the tissue.

### Mechanical Methods for Treating Tissue

The tissue may be mechanically treated using a homogenizer such as a: (1) Polytron® and/or rotor stator homogenizer (e.g., Tissue Tearor®); (2) dounce; (3) mortar and pestle; (4) tissue mill, mixer-mill, or bead-beater assembly (e.g., adding metal beads to tube with lysis buffer and the tissue sample and shaking), examples include TissueLyser® (Qiagen®) and the mini-beadbeater-8 (Biospec®); (5) blender (e.g., Waring® Blender); (6) spin column homogenizer (e.g., QIAshredder® from Qiagen®); and (7) sonicator (Cole-Parmer® Ultrasonic Processor.)

Further, the tissue may be mechanically comminuted to reduce the tissue into smaller pieces. Methods of comminuting the tissue include but are not limited to grinding, dicing, slicing, chopping up, comminuting, grinding with mortar and pestle, granulating, pressing, cubing, mincing, milling, grating, grading, crushing, rolling, shearing, dividing, hewing, or other method known in the art for changing a tissue from one size to another. The resultant size of tissue pieces can be a mixture of sizes or a collection of sizes. Mixtures, collections, and assortments of sizes need not be consistent in that the mixture, collection, and assortment can contain particles of different sizes. The resultant sized tissue pieces can also be uniform or substantially similar in size. After mechanical comminuting, the tissue may be further treated with enzymes, chemicals, or ultrasonic cavitation.

A good candidate for lipocavitation is someone looking for fat removal from a specific area such as the hips, thighs, buttocks, stomach or arms. The treatment does not generally result in overall weight loss, but an improved contour in the localized treatment area.

In the treatment the handpiece delivers low frequency ultrasound waves down into the subcutaneous or fatty layer of the skin, targeting the adipocytes. The minute vibrations produce tiny bubbles within the fat cells which disturb the outer membrane and allow tiny collections of fat to be expelled into the surrounding area, which then is removed via the body's natural energy and waste removal processes. This selective destruction of fat cells does not interfere with adjacent structures such as blood vessels and nerves and is therefore a very safe treatment. Lipocavitation is a painless procedure, though for some people there may be a little discomfort associated with the noise during treatment which ceases when the handpiece is no longer in contact with the skin. The tissue may be connective, muscle, nervous, adipose, and/or epithelial tissue. The lipocavitation may be done in situ in the patient and the resultant lysed cells comprising the stromal vascular fraction may be removed from the patient and the stromal vascular fraction isolated therefrom, using, for example, an ultrasonic flow cell device (e.g., Ultrasonic Mini Flow Cell from Hielscher Ultrasonics GmbH of Teltow, Germany.) In this method, the handpiece disrupts the blood vessels in a tissue, optionally connective, muscle, nervous, adipose, and/or epithelial tissue, and the disrupted tissue comprising the stromal vascular fraction is extracted and fed into an ultrasonic flow cell device (e.g., Ultrasonic Mini Flow Cell from Hielscher Ultrasonics GmbH of Teltow, Germany) and the stromal vascular fraction is isolated therefrom. In another method, the tissue, optionally connective, muscle, nervous, adipose, and/or epithelial tissue, is located in a patient and removed from the patient and fed into an ultrasonic flow cell device (e.g., Ultrasonic Mini Flow Cell from Hielscher Ultrasonics GmbH of Teltow, Germany) and the stromal vascular fraction is isolated therefrom.

The use of ultrasonic cavitation or lipocavitation is well known as a non-invasive treatment for the reduction of localized fat deposits. This method is used for people who are dissatisfied with a certain area of fatty deposits but who do not want to undergo any invasive surgical treatment like liposuction. It is performed as a walk in, walk out treatment and there is no lengthy recovery period as with surgical fat removal.

The ultrasonic cavitation methods described herein produce a stromal vascular fraction and specific cell types contained therein from tissue, optionally connective, muscle, nervous, adipose, and/or epithelial tissue, surgically obtained from the stromal or mesenchymal compartment of the body of a donor or derived from a liposuction derived aspirate.

Suitable tissue sources of SVF cells include, but are not limited to any fat-containing tissue, e.g., brown or white adipose tissue such as subcutaneous white adipose tissue. Typically, human tissue is obtained from a living donor using surgical excision or suction lumpectomy. The adipose tissue may be obtained from a pre-selected region on the subject, i.e., inguinal, retroperitoneal and gonadal, or any combination thereof.

The tissue, optionally connective, muscle, nervous, adipose, and/or epithelial tissue, treated with ultrasonic cavitation may be a lipoaspirate, adipose tissue sample removed during surgery, adipose tissue obtained from a non-living mammal, or adipose tissue obtained from a non-living animal.

SVF cells may be obtained from any animal (alive or dead) so long as stromal vascular fraction within the animal are viable. The tissue may be obtained from any post-mortem animal, for example an animal that is at least about 24 hours post-mortem. The tissue may be obtained from a human cadaver. The tissue may be obtained from a tissue bank, organ donation, or stored (e.g., frozen tissue). The tissue may be connective, muscle, nervous, adipose, and/or epithelial tissue.

The tissue may be stored in liquid nitrogen (i.e., −70° C.) or refrigerated (i.e., 4° C.).

The tissue may be obtained from a mammal including but not limited to alpacas, armadillos, capybaras, cats, camels, chimpanzees, chinchillas, cattle, dogs, elephants, goats, gorillas, hamsters, horses, humans, lemurs, llamas, mice, non-human primates, optionally monkeys, pigs, rats, sheep, shrews, squirrels, tapirs, and voles. The tissue may be harvested from a human. The tissue may be connective, muscle, nervous, adipose, and/or epithelial tissue.

Further, the inventor unexpectedly discovered that post-mortem sources of tissue may be used to obtain the stromal vascular fraction. For example, tissue taken from an animal up to 28 days post-mortem, the ultrasonic cavitation method described herein resulted in a high yield of viable cells. This surprising result has opened up an entirely new source of tissue for processing to obtain a stromal vascular fraction for cosmetic, research, and therapeutic uses (e.g., post-mortem sources).

The tissue, optionally connective, muscle, nervous, adipose, and/or epithelial tissue, may be fresh, frozen, or harvested post-mortem.

### Ultrasonic Cavitation

The ultrasonic cavitation methods described herein produce a stromal vascular fraction from vascular tissues contained in tissue which contains vascular tissue-derived stem cells and other cells found in the walls of blood vessels without the use of collagenase or another enzyme that cleaves collagen bonds.

The ultrasonic cavitation methods described herein produce a stromal vascular fraction from tissue, which method comprises treating tissue, optionally connective, muscle, nervous, adipose, and/or epithelial tissue, with ultrasonic cavitation under conditions whereby that the fat cells in the sample are exploded or lysed, and in addition under ultrasonication conditions whereby the blood vessels found in the fat are further lysed without adversely affecting the viability of stromal and mesenchymal stem cells contained therein. It has been found that by the judicious optimization of ultrasonication conditions as described herein, that ultrasonication methods may be used in the absence of protease treatment to release the desired stromal vascular or mesenchymal vascular cells from the blood vessels found in the tissue without adversely affecting the stromal and mesenchymal stem cells substantial lysis or degradation of the stromal and mesenchymal stem cells. Preferably, the methods will not include the addition of an enzyme that breaks down collagen such as a collagenase or other endopeptidase.

The ultrasonic cavitation methods described herein mechanically treat tissue, optionally connective, muscle, nervous, adipose, and/or epithelial tissue, ex vivo preferably in the absence of exogenous collagenase to lyse fat cells and the resultant sonically treated composition (from which the fat is removed) is then used to obtain a stromal vascular fraction which can be infused directly in patients in need thereof or it can be further processed to purify (and expand in culture if desired) desired cell types such as mesenchymal or stromal stem cells, endothelial cells, and other cells found in tissue. These fractions and cells may be used in patients such as for tissue reconstruction, tissue regeneration, wound healing, breast augmentation or reconstruction, in tissue fillers for plumping areas that have lost fullness, such as via aging or because of disease such as the face, lips, and the buttocks.

The use of an ultrasonic cavitation device having a probe that is placed into contact with the tissue, optionally connective, muscle, nervous, adipose, and/or epithelial tissue, and wherein the contact is sufficient (e.g., 1 minute to about 8 hours, more preferably about 5 minutes to about 1 hour) so as to explode or lyse most of the fat cells in the tissue and blood cells under conditions that release the stromal vascular fraction containing stromal and mesenchymal stem cells, endothelial precursors and other cell types contained therein without adversely affecting the viability and number of these cells from different samples. For example, the tissue sample may be treated with ultrasonic cavitation (sonication) for about 10 minutes. Further, the 10 minutes may be divided into two five minute periods where the probe is located towards the bottom of the sample for the first five minutes, sonication paused, the probe moved to the middle of the sample and continued for the remaining 5 minutes. A sonication probe of about 13 or 14 mm, optionally a 14 mm probe, may be used with a 200 W generator for the sonication device. Also, about 50-60 mL (cc) of tissue may be sonicated. In fact, as disclosed herein the present invention results in the recovery of increased numbers of viable stromal and mesenchymal stem cells (about ten-fold more) from adipose samples relative to prior art methods using collagenase or other enzymes.

The inventor surprisingly discovered that the unusually long period of sonication of about 10 minutes, optionally in two 5 minute periods, resulted in unexpectedly high numbers of viable cells. It is unexpected that this long period of ultrasonic cavitation would produce such large quantities of cells because sonication usually associated with disrupting/destroying cells. See Ultrasonic cell disruption from London South Bank University (2004) and Traditional Methods of Cell Lysis from Thermo Scientific (2012). Thus, the ultrasonic cavitation method described herein produces an unexpected results because one would not expect any cells to survive a 10 minute ultrasonic cavitation treatment.

Further, at the time of the invention, sonication was a common method used for cell lysis. Although a common form of sonication was known to be useful in lysing cells, the art gave no guidance of which parameters were critical and no direction of which combination of sonication parameters were useful for isolating stromal vascular fraction from tissue. Therefore, at the time the invention was made, it was impossible to choose the specific parameters useful in isolating intact, viable stromal vascular cells by ultrasonic cavitation. Thus, where the prior art gave no guidance on which parameters are critical, the inventor varied all of the parameters and tried each of a numerous possible choices until he arrived at a successful result. Indeed, the inventor tried over 100 combinations before approaching the successful method described herein.

After ultrasonic cavitation the exploded fat (at the top of the composition, e.g., supernatant) may be removed. The remaining fraction (at the bottom of the composition, e.g., pellet) may further purified or assayed (e.g., by flow cytometry) for the presence of desired cell types including stem and endothelial precursor cells, immune cells, osteoclasts, hematopoietic stem cells, and other cell types disclosed herein.

After ultrasonic cavitation the mesenchymal or stromal stem cells are isolated from the sample such as by flow cytometry or may be fractionated into different cell types using fluorescence activated cell sorting (FACS) based on cell surface antigens which are specific to adipose-derived stem cells or other cell lineages contained in tissue.

Thus, the ultrasonic cavitation method described herein provides for a method of isolating a stromal vascular fraction derived from tissue that does not comprise any exogenous collagenase or other exogenous enzymes.

The ultrasonic cavitation method described herein produce a stromal vascular fraction from tissue by treating tissue, optionally connective, muscle, nervous, adipose, and/or epithelial tissue, with ultrasonic cavitation under conditions whereby that the fat cells in the sample are exploded or lysed thereby releasing the stromal vascular or mesenchymal vascular cells from the tissue and which preferably does not include the addition of an enzyme that breaks down collagen such as a collagenase or other endopeptidase. The tissue may be derived from any mammal but preferably is from a living or non-living donor. The tissue may be obtained via liposuction surgery, aspiration of fat after these procedures or isolated by other surgical methods.

The method described herein may use an ultrasonic cavitation device having a probe that is placed into contact with the tissue and wherein the contact is for a sufficient time (e.g., about 10 minutes) so as to explode or lyse most of the fat cells in the tissue and release the stromal vascular fraction containing stromal and mesenchymal stem cells, endothelial precursors and other cell types.

Ultrasonic cavitation devices known in the art may be used. One suitable selection is the Vibra-Cell® device which is a technologically advanced high intensity ultrasonic processor. This device can safely process a wide range of organic and inorganic materials—from microliters to liters. Other devices which may be used include HIELSCHLER SONIC 200®, and SONIC 200®. The ultrasonic cavitation devices may have a 200 W generator and a rod size of about 13-14 mm. The ultrasonic cavitation devices may have a 200 W generator and a rod size of about 14 mm. The ultrasonic cavitation device may have a 200 W-500 W generator.

After the tissue containing blood vessels is treated using the ultrasonic cavitation device the exploded fat (at the top of the composition) may be removed and the remaining stromal or mesenchymal vascular fraction from the lysed or exploded blood vessels may be further purified or assayed (e.g., by flow cytometry) for the presence of desired cell types including stem and endothelial precursor cells. This may be effected by known methods including flow cytometry or fractionation into different cell types using fluorescence activated call sorting (FACS), e.g., based on cell surface antigens which are specific to adipose-derived stem cells or other cell lineages contained in tissue. Suitable antigens and markers are disclosed herein.

The separated tissue optionally can be washed with any suitable physiologically-compatible solution, such as phosphate buffer saline (PBS) or normal saline. Using the exemplified methods, washing is not required, i.e., simply place the sonication rod into adipose sample or lipoaspirate and turn on the sonication device thereby processing the sample with the ultrasonic cavitation device. After treatment three layers form after settlement of the dissociated tissue. The top layer is a free lipid (fat) layer. The middle layer includes the lattice and adipocyte aggregates. The bottom layer or cell pellet which is produced after the treated composition is allowed to settle or is centrifuged and contains the stromal vascular fraction (SVF). The tissue may be connective, muscle, nervous, adipose, and/or epithelial tissue.

The cellular fraction of the bottom layer may be infused into a subject or may be further concentrated into a pellet by any suitable method, e.g., centrifugation, and retained for further processing. If desired the stromal vascular fraction (SVF) may be resuspended and can be further washed in physiologically compatible buffer (e.g., phosphate-buffered saline [PBS]), centrifuged, and resuspended one or more successive times to achieve greater purity. The cells of the washed and resuspended pellet may also be plated.

Morphological, biochemical or molecular-based methods may be used to identify or isolate the cells in the stromal vascular fraction (SVF). In one aspect, SVF cells are isolated based on cell size and granularity since SVF cells are small and agranular. Alternatively, because stem cells tend to have longer telomeres than differentiated cells, SVF cells can be isolated by assaying the length of the telomere or by assaying for telomerase activity.

Alternatively, SVF cells can be separated from the other cells of the pellet immunohistochemically by selecting for adipose derived stem cell (ADSC)-specific cell markers using suitable materials and methods, e.g., panning, using magnetic beads, or affinity chromatography. Suitable markers include any of the markers disclosed in this application or any combination thereof.

### Ultrasonic Cavitation Conditions

The ultrasonic cavitation may be performed at a frequency of about 20-30 kHz. The ultrasonic cavitation may be performed at a frequency of about 20, 21, 22, 23, 24, 24, or 25 kHz. The ultrasonic cavitation may be performed at a frequency of about 24 kHz. The ultrasonic cavitation may be performed at a frequency of about 20-23 kHz or 23-25 kHz.

The ultrasonic cavitation may be performed using an ultrasonic probe of about 10-15 mm, about 13 or 14 mm probe. The ultrasonic cavitation may be performed using an ultrasonic probe of about 13 mm. The ultrasonic cavitation may be performed using an ultrasonic probe of about 14 mm.

The ultrasonic cavitation device can be an ultrasonic cup horn or an ultrasonic flow cell. The ultrasonic cavitation device may be VialTweeter available from Hielscher which can be used to sonicate a test tube, bottle, or beaker up to 500 mL. The ultrasonic cavitation device may be a handheld or stand-mounted sonicator. The ultrasonic cavitation device may be a flow cell device that can sonicate material in continuous flow, e.g., for at about 20-200 mL per minute. Further, the ultrasonic cavitation device may be a microtiter plate sonication device. See, e.g., UIP250MTP Sonication of Microtiter Plates from Hielscher.

The ultrasonic cavitation device may be an ultrasonic mini flow cell device, for example available from Hielscher. In an ultrasonic mini flow cell device ultrasound energy is transmitted into small volumes via a titanium sonotrode immersed into the liquid. An advantage of using an ultrasonic mini flow cell device is that no contact between the sonotrode and the sample. The major advantage of this is that in traditional ultrasonic cavitation where the ultrasonic rod in placed in the sample there is the risk that microscopic pieces of the metal rod may erode into the sample due to self-destruction of the metal rod by the ultrasonic energy. This eliminates the possibility of any contamination from fine titanium particles shed by the titanium sonotrode.

This can be a closed system that can be done in the lab under cGTPs or at patient's bedside and hooked directly to the liposuction cannulae. For example, tissue may be harvested using an indirect sonicator and transmitted to an ultrasonic flow cell device where the tissue is subjected to ultrasonic cavitation to allow for the isolation of stromal vascular fraction (SVF).

In an ultrasonic flow cell device comprising a steel jacket coupled to a pressurized water intake and a pressured water out-take wherein the pressurized water runs through the interior of the steel jacket over a glass conduit that runs through the center of the steel jacket with seals for a water-tight seal. The steel jacket is coupled to a transducer and sonotrode. Lipoaspirate is provided to an product input that runs through the steel jacket opposite to the direction of pressurized water. A transducer and sonotrode provides ultrasonic energy.

In an assemblage comprising an ultrasonic flow cell device coupled to an indirect sonicator. Tissue, optionally connective, muscle, nervous, adipose, and/or epithelial tissue, may be harvested using an indirect sonicator from a patient and run through an ultrasonic flow cell device that allows for the isolation of stromal vascular faction cells using the methods described herein.

The tissue, optionally connective, muscle, nervous, adipose, and/or epithelial tissue, sample size subjected to ultrasonic cavitation may be about 5-200 cc (mL), 10-100 cc (mL), 20-80 cc (mL), 40-60 cc (mL), or 50-60 cc (mL). The tissue sample size subjected to ultrasonic cavitation may be about 40, 45, 50, 50, 55, or 60 cc (mL).

The ultrasonic cavitation may be performed at a temperature at between about 0° C. and 25° C. The ultrasonic cavitation may be performed at a temperature at about 4° C., for example in an ice bath, ice bucket, or cold water. The ultrasonic cavitation may be performed at a temperature at about 23° C., for example in an water bath.

The tissue, optionally connective, muscle, nervous, adipose, and/or epithelial tissue, may be sonicated in a vessel of about 60 mm in size, preferably a conical polypropylene tube.

The ultrasonic cavitation may be performed using a 200 W generator. The ultrasonic generator can be a 200-2000 W generator, optionally a 200 W or 2000 W generator. The power can range from about 2-50 KW.

The ultrasonic cavitation may be performed at a duration of about 1-60, 1-30, or 1-10 minutes. For example, the ultrasonic cavitation may be for about 10 minutes in two 5 minute periods. The tissue may be sonicated for 1-60, 1-30, or 1-10 minutes. The tissue may be sonicated for 10 minutes in two 5 minute periods. The ultrasonic probe may be inserted towards the bottom of the tube. The ultrasonic probe may be moved, after 5 minutes of sonication, towards the middle of the tube, for an additional 5 minutes.

The ultrasonic cavitation may be performed by using a 200 W generator, a 13-14 mm rod, optionally a 13 or 14 mm rod for two 5 minute periods, optionally consecutive, where the first 5 minute period of ultrasonic cavitation is at the lower portion of the sample and the second 5 minute period of ultrasonic cavitation is about half-way in the sample. The container may be a 60 cc tube size, 28 mm diameter and 110 mm length and the sample may be about 50-60 cc (mL).

The ultrasonic cavitation method may result in a yield of about 2,000,000 up to about 22,000,000 stromal vascular cells per mL of tissue. The ultrasonic cavitation method may result in a yield of about 1×10⁶, 2×10⁶, 3×10⁶, 4×10⁶, 5×10⁶, 6×10⁶, 7×10⁶, 8×10⁶, 9×10⁶, 10×10⁶, 11×10⁶, 12×10⁶, 13×10⁶, 14×10⁶, 15×10⁶, 16×10⁶, 17×10⁶, 18×10⁶, 19×10⁶, 20×10⁶, 21×10⁶, or 22×10⁶ stromal vascular cells per mL of tissue. The ultrasonic cavitation method may result in a yield of about 1×10⁷, 2×10⁷, 3×10⁷, 4×10⁷, 5×10⁷, 6×10⁷, 7×10⁷, 8×10⁷, 9×10⁷, or 10×10⁷ stromal vascular cells per mL of tissue. The ultrasonic cavitation method may result in a yield of at least about 1×10⁷ stromal vascular cells per mL of tissue.

The ultrasonic cavitation method may result in a cell yield with stromal vascular cells with at least about 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% viability. The ultrasonic cavitation method may result in a cell yield with stromal vascular cells with at least about 80%, 85%, 90%, or 95% viability.

The tissue, optionally connective, muscle, nervous, adipose, and/or epithelial tissue, may be obtained from a non-living animal, including mammals. The tissue may be obtained from a mammal at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 hours post-mortem. The tissue may be obtained from a mammal at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 days post-mortem.

Also, the stromal vascular fraction cells may be isolated from human tissue, optionally connective, muscle, nervous, adipose, and/or epithelial tissue. First the tissue is washed with PBS, cut it into about 1-2 mm pieces and digest with 1 mg/ml collagenase in Krebs-ringer-bicarbonate buffer for 40 minutes at 37° C. After the digestion, the cells are filtrated first through 100 μm and 40 μm, wash with ice cold Kreb buffer twice the initial volume. The sample is then subject to two centrifugation steps at about 300 RCF at about 5 minutes resulting in a supernatant and pellet. The supernatant largely comprises fat which may be removed and the pellet comprising the SVF cells may be washed with PBS and the centrifugation repeated.

The isolated stromal vascular fraction or isolated cells are preferably administered into a patient in need thereof. For example the stem cells are used to promote wound healing, breast augmentation or reconstruction, tissue engineering, or other applications.

### Indirect Ultrasonic Cavitation

The indirect ultrasonic cavitation can be apply directly externally to the fat ie the UC can be directed through the patient's skin and the blood vessels in the tissue are destroyed to release stromal vascular fraction. An ultrasonic cavitation rod can be inserted through a puncture or incision in the skin into the patient's tissue and directly destroy the blood vessels to release SVF. Further, tissue, optionally connective, muscle, nervous, adipose, and/or epithelial tissue, may be located using ultrasound device and then removed using indirect cavitation device and subject to ultrasonic cavitation to release the SVF. The SVF may be further isolated, centrifuged, and used in therapeutic methods, for example autologous or allogeneic transplantation.

For the isolation of stromal vascular fraction from tissue, the ultrasonic cavitation may be applied to the external of the patient's body, in effect subjecting the tissue, optionally connective, muscle, nervous, adipose, and/or epithelial tissue, to ultrasonic cavitation in situ. The resultant lysed tissue, optionally adipose tissue, comprising the stromal vascular fraction may then be extracted and the stromal vascular fraction isolated from the extract.

The ultrasonic cavitation device may be inserted directly into the patient's body subjecting the tissue, optionally connective, muscle, nervous, adipose, and/or epithelial tissue, ultrasonic cavitation. The resultant lysed tissue, optionally connective, muscle, nervous, adipose, and/or epithelial tissue, comprising the stromal vascular fraction may then be extracted and the stromal vascular fraction isolated from the extract. This can be a closed system performed in the lab under cGTPs or at patient's bedside and coupled to the liposuction cannulae.

Further, the tissue, optionally connective, muscle, nervous, adipose, and/or epithelial tissue, may be removed using a liposuction cannula and then subject to indirect ultrasonic cavitation. The tissue may be placed in a metal vessel where the ultrasonic probe impinges sound waves through water bath underneath the sample (e.g., no contact between the probe and the sample). This prevents possible contamination of the sample by microscopic metal fragments shed from the probe.

Additionally, the tissue, optionally adipose tissue, may be extracted from the patient and subjected to indirect ultrasonic cavitation to lysing the blood vessels and releasing the stromal vascular fraction. The lysed tissue may then be fed into a centrifuge, optionally a filter, to remove the lysed tissue and isolate the stromal vascular fraction.

The tissue, optionally adipose tissue, may be subjected to ultrasonic cavitation in the patient in situ, lysing the blood vessels and releasing the stromal vascular fraction. The released stromal vascular fraction may then be isolated from the patient directly and used in therapeutic methods, uses, and compositions described herein.

A patient may be selected and tissue, optionally adipose tissue, subjected to mechanical, chemical, and/or enzymatic treatment to release the stromal vascular fraction. The method for releasing the stromal vascular fraction comprises subjecting said tissue to ultrasonic cavitation, wherein the cells in the tissue and blood vessels in the tissue are lysed, thereby dissociating or releasing substantial numbers of intact stromal vascular fraction cells from the lysed blood vessels contained in the ultrasonicated tissue while substantially maintaining the viability of the cells constituting the stromal vascular fraction. The tissue may be subjected to ultrasonic cavitation by bringing the tissue into contact with a powerful ultrasonic cup horn, ultrasonic probe (rod), ultrasonic flow cell, or ultrasonic microtiter plate. The stromal vascular fraction may be extracted from the patient in situ. The method may further comprises removing the lysed tissue comprising the lysed blood vessels and the stromal vascular fraction, optionally adipose tissue. Also, the target tissue may be identified and/or targeting using ultrasound.

The ultrasonic cavitation may be applied directly externally to the tissue, optionally applied to the patient's skin and the blood vessels are lysed to release the stromal vascular fraction. To isolate the stromal vascular fraction, the ultrasonic cavitation rod may be inserted through a puncture or incision in the skin into the patient's tissue and lyse the blood vessels to release stromal vascular fraction. The tissue located using ultrasound device may then be removed using indirect cavitation device and subject to ultrasonic cavitation to release the stromal vascular fraction. The tissue may be, optionally located using ultrasound device, removed subject to ultrasonic cavitation by an ultrasonic flow cell device, optionally, an Ultrasonic Mini Flow Cell, to release the stromal vascular fraction, optionally, wherein the stromal vascular fraction is further isolated.

### Stromal Vascular Fraction

The inventor surprisingly discovered that tissues, optionally connective, muscle, nervous, adipose, and/or epithelial tissue, e.g., derived from surgical excision or aspirated via liposuction may be treated by ultrasonic cavitation for a sufficient amount of time to explode or lyse the fat cells and the blood vessels contained therein and thereby release stromal vascular fraction cells contained within the blood vessels in the tissue including stromal and mesenchymal stem cells, endothelial precursor cells, and other cell types which constitute the stromal vascular fraction.

The invention provides a novel method of obtaining a stromal vascular fraction from tissue, optionally connective, muscle, nervous, adipose, and/or epithelial tissue, that does not include the use of collagenase or other enzymes to digest the collagen bonds that hold together the tissue. While collagenase works well for this purpose, and indeed is conventionally used by those skilled in the art to degrade collagen and separate the tissue into discrete cells, the use of this enzyme may be disadvantageous for cellular products that are to be used in humans, e.g., cells or cell fractions which are to be used in tissue reconstruction or regeneration, e.g., breast reconstruction procedures, cosmetic skin rejuvenation or usage in cosmetic tissue fillers that are used during plastic surgery. Particularly the FDA may consider that the use of this enzyme (to derive desired cells) results in a “maximally manipulated” cellular product. This is disadvantageous as the use of collagenase would potentially place stromal or mesenchymal vascular cells derived from tissue in a category that requires drug approval, even if the cell fraction is to be used cosmetically and not clinically.

Also, the use of enzymes such as collagenase is further disfavored as these enzymes result in more cell death, thereby reducing the number of the desired cells which are isolatable, and further this results in more cellular debris, thereby resulting in a less useful cell product, especially if the cells are to be used therapeutically.

In collagenase protocol requires washing out blood. The blood contains hematopoietic stem cells, blood is retained, red blood cells are destroyed but hematopoietic stem cells are surprisingly retained. This has the unexpected result of producing a stromal vascular faction product with better therapeutic properties than prior stromal vascular fraction products. Further, the stromal vascular fraction products isolated by the methods described herein do not have any enzymes which must be remove prior to clinical use.

In collagenase protocol requires washing out blood. The blood contains hematopoietic stem cells, blood is retained, red blood cells are destroyed but hematopoietic stem cells are surprisingly retained. This has the unexpected result of producing a stromal vascular faction product with better therapeutic properties than prior stromal vascular fraction products. Further, the stromal vascular fraction products isolated by the methods described herein do not have any enzymes which must be remove prior to clinical use.

The SVF cells obtained by the ultrasonic cavitation methods described in U.S. Patent Application Publication No. 2012/0164113 are different than the SVF cell product produced using enzymes, e.g., collagenase, in part because the methods described herein do not wash out the blood. Thus, all of the hematopoietic elements are present in the stromal vascular fraction cells prepared by the ultrasonic cavitation methods described in U.S. Patent Application Publication No. 2012/0164113 whereas they are not in SVF cells produced by prior art methods which use enzymes. Further, stromal vascular cells (SVF) isolated by these ultrasonic cavitation methods retain hematopoietic factors. Also, in samples digested by enzymes it is necessary to wash out the blood components before processing (e.g. hematopoietic stem cells, glucose). The stromal vascular fraction cells prepared by the ultrasonic cavitation methods described in U.S. Patent Application Publication No. 2012/0164113 retain these important components. The presence of the blood components, specifically glucose, gives the stromal vascular fraction cells prepared by the ultrasonic cavitation methods described in U.S. Patent Application Publication No. 2012/0164113 a longer viability when stored at room temperature.

Additionally, the stromal vascular fraction cells prepared by the ultrasonic cavitation methods described in U.S. Patent Application Publication No. 2012/0164113 are different than the SVF cell product produced using enzymes, e.g., collagenase, in part because the methods described herein do not wash out the extracellular matrix (ECM). The extracellular matrix (ECM) is the extracellular part of animal tissue that usually provides structural support to the animal cells in addition to performing various other important functions. The extracellular matrix is the defining feature of connective tissue in animals. Extracellular matrix includes the interstitial matrix and the basement membrane. Interstitial matrix is present between various animal cells (i.e., in the intercellular spaces). Gels of polysaccharides and fibrous proteins fill the interstitial space and act as a compression buffer against the stress placed on the ECM. Basement membranes are sheet-like depositions of ECM on which various epithelial cells rest. Due to its diverse nature and composition, the ECM can serve many functions, such as providing support, segregating tissues from one another, and regulating intercellular communication. The extracellular matrix regulates a cell's dynamic behavior. In addition, it sequesters a wide range of cellular growth factors, and acts as a local depot for them. Changes in physiological conditions can trigger protease activities that cause local release of such depots. This allows the rapid and local growth factor-mediated activation of cellular functions, without de novo synthesis. Formation of the extracellular matrix is essential for processes like growth, wound healing and fibrosis. Therefore, the stromal vascular fraction cells prepared by the ultrasonic cavitation methods described in U.S. Patent Application Publication No. 2012/0164113 comprise extracellular matrix components, unlike SVF prepared by enzymatic treatment, and thus may have better healing properties.

TABLE 1
Comparison of SVF prepared by methods described
herein and collagenase methods.

| UltraSonic Caviation | Collagenase Enzyme prepared SVF |
|---|---|
| 20-33 × 10⁶ cell/mL | 3-8 × 10⁶ cell/mL |
| 5-8% dead cells | 15-30% cells |
| 1-3% debris | 5-15% debris |
| total volume of cells 40-50 mL | total volume of cells 20 mL |
| total dose 500 million to | total dose 50-70 million cells |
| 1.2 billion cells | |

Accordingly it is desirable to provide alternative methods, e.g., mechanical methods, that produce a stromal vascular fraction (containing mesenchymal stem cells, endothelial cells, and other cells found in adipose tissues) which is suitable for administration to patients via local or systemic administration such as via injection, infusion, topical administration, or which is administered in association with implants, matrices, tissue fillers, wherein the adipose tissue derived composition does not include collagenase and is not “maximally manipulated” according to the FDA.

The inventor has surprisingly discovered that tissues, optionally connective, muscle, nervous, adipose, and/or epithelial tissue (e.g., derived from surgical excision or aspirated via liposuction), may be treated ex vivo or in situ by ultrasonic cavitation for a sufficient amount of time to explode or lyse the tissue cells and the blood vessels contained therein thereby releasing the stromal vascular fraction cells contained within the outer layer of blood vessel walls contained in the tissue including mesenchymal stem cells, endothelial precursor cells, and other cell types which constitute the “stromal vascular fraction.” The inventor has unexpectedly found that the treatment of tissue by use of ultrasonic cavitation under appropriate conditions such as exemplified in the working examples, not only explodes or lyses the tissue cells, but further explodes or lyses the blood vessels contained therein, without adversely affecting the viability of stromal and mesenchymal stem cells, thereby releasing high numbers of viable stromal and mesenchymal stem cells, endothelial precursor cells, and other cell types which constitute the “stromal vascular fraction” which stromal and mesenchymal stem cells may be recovered and used in desired cosmetic or therapeutic methods wherein these cells are of beneficial value. This was unexpectedly expanded to cover any vascularized tissue, e.g., any tissue that comprises blood vessels may be subjected to ultrasonic cavitation or other means to isolate a stromal vascular fraction.

The stromal vascular fraction may comprise stem and other cells that express at least one protein selected from the group consisting of CD13, CD14, CD29, CD31, CD34, CD36, CD44, CD45. CD49d, CD54, CD58, CD71, CD73, CD90, CD105, CD106, CD151 and SH3, or CD13, CD29, CD34, CD36, CD44, CD49d, CD54, CD58, CD71, CD73, CD90, CD105, CD106, CD151 and SH3 and/or CD31, CD45, CD117 and CD146. Further, the stromal vascular fraction may comprise stem and other cells that do not express CD56.

The stromal vascular fraction may comprise stem and other cells that express at least one protein selected from the group consisting of CD3, CD4, CD14, CD15, CD16, CD19, CD33, CD38, CD56, CD61, CD62e, CD62p, CD69, CD104, CD135 and CD144, and does not express CD3, CD4, CD14, CD15, CD16, CD19, CD33, CD38, CD56, CD61, CD62e, CD62p, CD69, CD104, CD135 and CD144. Also, the stromal vascular fraction may comprise stem and other cells that express CD49d but do not express CD56.

Cells contained therein and markers isolatable from the stromal vascular fraction of tissue according to the methods described herein include by way of example mesenchymal stem cells, hematopoietic cells, endothelial precursor cells (EPC), hematopoietic stem cells, platelets, Kupffer cells, osteoclasts, megakaryocytes, granulocytes, NK cells, endothelial precursor or progenitor cells, CD34+ cells, CD29+ cells, CD166+ cells, Thy-1+ or CD90+ stem cells, CD44+ cells, immune cells such as monocytes, leukocytes, lymphocytes, B and T cells, NK cells, macrophages, neutrophil leukocytes, neutrophils, neutrophil granulocytes, and the like including immune and other cells that express one or more of the following markers: CD3, CD14 (macrophage marker), CD19, CD20 (B cell marker), CD29 (integrin unit) CD31 (endothelial, platelet, macrophage, Kupffer cell, granulocyte, T/NK cells, lymphocytes, megakaryocytes, osteoclasts, neutrophils, et al.), CD44 (Hyaluronic acid receptor) CD45 (B and T cell marker), C56, CD73 (lymphocyte differentiation marker), CD105 et al. Also, it includes cells expressing any of the markers disclosed in this application or any combination of these markers.

The stromal vascular fraction may comprise adipose-derived stem cells that express at least one protein selected from the group consisting of CD13, CD29, CD34, CD36, CD44, CD49d, CD54, CD58, CD71, CD73, CD90, CD105, CD106, CD151 and SH3, or CD13, CD29, CD34, CD36, CD44, CD49d, CD54, CD58, CD71, CD73, CD90, CD105, CD106, CD151 and SH3 and/or CD31, CD45, CD117 and CD146 and will not express CD56.

The stromal vascular fraction may comprise stem cells that express at least one protein selected from the group consisting of CD3, CD4, CD14, CD15, CD16, CD19, CD33, CD38, CD56, CD61, CD62e, CD62p, CD69, CD104, CD135 and CD144, and does not express CD3, CD4, CD14, CD15, CD16, CD19, CD33, CD38, CD56, CD61, CD62e, CD62p, CD69, CD104, CD135 and CD144 or expresses CD49d and does not express CD56.

While the stromal vascular fraction (SVF) or isolated cells derived from tissue may be used directly for treatment, alternatively the cells may be expanded in culture such that a single milliliter of tissue yields over 400,000 cells. Aust, et al. (2004) Cytotherapy 6: 1-8. Undifferentiated human adipocyte cells express a distinct immunophenotype based on flow cytometric analyses and, following induction, produce additional adipocyte specific proteins. Aust, et al. (2004) Cytotherapy 6: 1-8; Gronthos, et al. (2001) J. Cell Physiol. 189: 54-63; Halvorsen, et al. (2001) Metabolism 50: 407-413; Sen, (2001) J. Cell. Biochem. 81: 312-319; Zuk, et al. (2002) Mol. Biol. Cell. 13: 4279-4295. Human adipose-derived adult stem cells (huASCs) display multipotentiality, with the capability of differentiating along the adipocyte, chondrocyte, myogenic, neuronal, and osteoblast lineages. Aust, et al. (2004) Cytotherapy 6: 1-8; Gronthos, et al. (2001) J. Cell Physiol. 189: 54-63; Halvorsen, et al., (2001) Metabolism 50: 407-413; Sen (2001) J. Cell. Biochem. 81: 312-319; Zuk, et al. (2002) Mol. Biol. Cell. 13: 4279-4295; Ashjian, et al. (2003) Plast. Reconstr. Surg. 111: 1922-19231; Awad, et al. (2003) Tissue Engineering 9: 1301-1312; Awad, et al. (2004) Biomaterials 25: 3211-3222; Halvorsen, et al. (2001) Tissue Eng. 7: 729-741; Hicok, et al. (2004) Tissue Engineering 10: 371-380; Mizuno, et al. (2002) Plast. Reconstr. Surg. 109: 199-209; Safford, et al. (2002) Biochem. Biophys. Res. Commun. 294: 371-379; Safford, et al. (2004) Experimental Neurology 187: 319-328; Wickham, et al. (2003) Clin. Orthop. 412: 196-212; Winter, et al. (2003) Arthritis Rheum. 48: 418-429; Zuk, et al. (2001) Tissue Eng. 7: 211-28. In the presence of dexamethasone, insulin, isobutylmethylxanthine and a thiazolidinedione, the undifferentiated human adipocyte cells undergo adipogenesis as evidenced by the fact that between 30% to 80% of the cells, based on flow cytometric methods, accumulate lipid vacuoles, which can be stained for neutral lipid with Oil Red O dye. Halvorsen, et al. (2001) Metabolism 50: 407-413; Sen, et al. (2001) J. Cell. Biochem. 81: 312-319. Further, the stromal vascular fraction isolated by the methods described herein may be cultured.

Methods to isolate and expand SVF cells are known in the art and described, for example in U.S. Pat. Nos. 6,391,297 B1; 6,777,231B1; Burris, et al. (1999) Mol Endocrinol 13:410-7; Erickson, et al. (2002) Biochem Biophys Res Commun. 290(2):763-9; Gronthos et al. (2001) Journal of Cellular Physiology, 189:54-63; Halvorsen, et al. (2001) Metabolism 50:407-413; Halvorsen, et al. (2001) Tissue Eng. 7(6):729-41; Harp, et al. (2001) Biochem Biophys Res Commun 281:907-912; Saladin, et al. (1999) Cell Growth & Diff 10:43-48; Sen et al. (2001) Journal of Cellular Biochemistry 81:312-319; Zhou, et al. (1999) Biotechnol. Techniques 13: 513-517; Zuk et al. (2001) Tissue Eng. 7: 211-228.

Mesenchymal stem cells isolated from the SVF obtained using the methods described herein may be cultured without differentiation using standard cell culture media, referred to herein as control medium (e.g., DMEM, typically supplemented with 5-15% serum (e.g., fetal bovine serum, horse serum). The stem cells can be passaged at least five times or even more than twenty times in this or similar medium without differentiating to obtain a substantially homogeneous population of SVF cells. The SVF cells can be identified by phenotypic identification. To phenotypically separate the SVF cells, the cells are plated at any suitable density which may be anywhere from between about 100 cells/cm² to about 100,000 cells/cm² (e.g., about 500 cells/cm² to about 50,000 cells/cm², between about 1,000 cells/cm² to about 20,000 cells/cm²).

After passaging for several days, SVF cells initially plated at lower densities (at less than 500 cells/cm², or alternatively, less than about 300 cells/cm² or alternatively, at less than 100 cells/cm²) can be used to obtain a clonal population of SVF cells by any suitable method such as by physically picking and seeding cells into separate plates (such as the well of a multi-well plate). Alternatively, the stem cells can be subcloned into a multi-well plate at a statistical ratio for facilitating placing a single cell into each well (e.g., from about 0.1 to about 1 cell/well or even about 0.25 to about 0.5 cells/well, such as 0.5 cells/well). Cloning can be facilitated by the use of cloning rings. See MacFarland (2000) Methods in Cell Sci. 22:63-66. Alternatively, where an irradiation source is available, clones can be obtained by permitting the cells to grow into a monolayer and then shielding one and irradiating the rest of the cells within the monolayer. The surviving cell then will grow into a clonal population. Alternatively, plated cells can be diluted to a density of 10 cells/ml and plated on Nunclon 96-well plates (Nalge Nunc International). Only wells that contain a single cell at the outset of the culture period are assayed for colony formation. Clones are detectable by microscopy after 4 to 5 days.

An exemplary culture condition for cloning stem cells comprises about 213 F12 medium+20% serum (preferably fetal bovine serum) and about 113 standard medium that has been conditioned with stromal cells or 15% FBS, 1% antibiotic/antimycotic in F-12/DMEM [1:1]) (e.g., cells from the stromal vascular fraction of liposuction aspirate, the relative proportions can be determined volumetrically).

The white blood cells (WBCs), as known as leukocytes, may be removed from the allogeneic SVF compositions described herein. The concentration of white blood cells in human whole blood is about 4,000-11,000 white blood cells per microliter. The WBC count in a microliter of the allogeneic SVF composition may be between about 1,000-4,000; about 1,000-4,000; about 500-2,000; about 1,500-2,000; about 200-1,000; about 200-500; about 500-750; or 75-100 white blood cells per microliter. The SVF composition may be essentially free of white blood cells. The SVF composition may comprise less than 1,000 WBCs.

### Cryopreserved Preparations of Stromal Vascular Fraction Cells

The stromal vascular fraction cells may be frozen for storage. The stromal vascular fraction cells may be stored by any appropriate method known in the art (e.g., cryogenically frozen) and may be frozen at any temperature appropriate for storage of the cells. For example, the cells may be frozen at about −20° C., −80° C., −120° C., −130° C., −135° C., −140° C., −150° C., −160° C., −170° C., −180° C., −190° C., −196° C., at any other temperature appropriate for storage of cells. Cryogenically frozen cells may be stored in appropriate containers and prepared for storage to reduce risk of cell damage and maximize the likelihood that the cells will survive thawing. The stromal vascular fraction cells may be cryopreserved immediately following differentiation, following in vitro maturation, or after some period of time in culture. The stromal vascular fraction cells may also be maintained at room temperature, or refrigerated at, for example, about 4° C.

Similarly provided are methods of cryopreserving stromal vascular fraction cells. The stromal vascular fraction cells may be harvested, washed in buffer or media, counted, concentrated (via centrifugation), formulated in freezing media (e.g., 90% FBS/10% DMSO), or any combination of these steps. For example, the stromal vascular fraction cells may be seeded in several culture vessels and serially expanded. As the stromal vascular fraction cells are harvested and maintained in FBS at about 4° C. while several flasks of stromal vascular fraction cells are combined into a single lot. The stromal vascular fraction cells may be also washed with saline solution (e.g., DPBS) at least 1, 2, 3, 4, or 5 times. The information on the label may include the type of cell (e.g., stromal vascular fraction cells), the lot number and date, the number of cells (e.g., 1×10⁶ cells/mL), the expiration date (e.g., recommended date by which the vial should be used), manufacture information (e.g., name and address), warnings, and the storage means (e.g., storage in liquid nitrogen).

Cryopreserved stromal vascular fraction (SVF) cell preparations described herein may comprise at least about 50,000-100,000 stromal vascular fraction cells. The cryopreserved stromal vascular fraction cell preparations may also comprise at least about 20,000-500,000 SVF cells. Also, the cryopreserved SVF cell preparations may comprise at least about 5,000, 10,000, 20,000, 30,000, 40,000, 50,000, 60,000, 75,000, 80,000, or 100,000 SVF cells. The cryopreserved SVF cell preparations may comprise at least about 1,000, 2,000, 3,000, 4,000, 5,000, 10,000, 20,000, 30,000, 40,000, 50,000, 60,000, 75,000, 80,000, 100,000, or 500,000 SVF cells. The cryopreserved SVF cell preparations may comprise at least about 1,000, 2,000, 3,000, 4,000, 5,000, 6,000, 7,000, 8,000, 9,000, 1×10⁴, 2×10⁴, 3×10⁴, 4×10⁴, 5×10⁴, 6×10⁴, 7×10⁴, 8×10⁴, 9×10⁴, 1×10⁵, 2×10⁵, 3×10⁵, 4×10⁵, 5×10⁵, 6×10⁵, 7×10⁵, 8×10⁵, 9×10⁵, 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁷, 3×10⁹, 4×10⁹, 5×10⁹, 6×10⁹, 7×10⁹, 8×10⁹, or 9×10⁹ SVF cells. The SVF cells of the cryopreserved SVF cell preparations may be mammalian SVF cells, including human SVF cells.

Further, the cryopreserved SVF cell preparations described herein may comprise at least about 50,000-100,000 SVF cells/mL. The cryopreserved SVF cell preparations may also comprise at least about 20,000-500,000 SVF cells/mL. Also, the cryopreserved SVF cell preparations may comprise at least about 5,000, 10,000, 20,000, 30,000, 40,000, 50,000, 60,000, 75,000, 80,000, and 100,000 SVF cells/mL. The cryopreserved SVF cell preparations may comprise at least about 1,000, 2,000, 3,000, 4,000, 5,000, 10,000, 20,000, 30,000, 40,000, 50,000, 60,000, 75,000, 80,000, 100,000, or 500,000 SVF cells/mL. The cryopreserved SVF cell preparations may comprise at least about 1,000, 2,000, 3,000, 4,000, 5,000, 6,000, 7,000, 8,000, 9,000, 1×10⁴, 2×10⁴, 3×10⁴, 4×10⁴, 5×10⁴, 6×10⁴, 7×10⁴, 8×10⁴, 9×10⁴, 1×10⁵, 2×10⁵, 3×10⁵, 4×10⁵, 5×10⁵, 6×10⁵, 7×10⁵, 8×10⁵, 9×10⁵, 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁷, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10, or 9×10¹⁰ SVF cells/mL. The SVF cells of the cryopreserved SVF cell preparations may be mammalian SVF cells, including human SVF cells.

The SVF cells of the invention may be recovered from storage following cryopreservation. The SVF cells recovered from cryopreservation also maintain their viability and differentiation status. For example, at least about 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% of the SVF cells may retain viability and differentiation following cryopreservation. Further, the SVF cells of the invention may be cryopreserved and maintain their viability after being stored for at least about 1, 2, 3, 4, 5, 6, or 7 days. The SVF cells of the invention may also be cryopreserved and maintain their viability after being stored for at least about 1, 2, 3, 4, 5, 6, 7, 8, 9, 10, 11, or 12 months. The SVF cells of the invention may be cryopreserved and maintain their viability after being stored for at least about 1, 2, 3, 4, 5, 6, or 7 years. The cryopreservation preparation comprising SVF cells may be substantially free of DMSO.

The invention also provides a method of cryopreserving stromal vascular fraction cells comprising (a) isolating stromal vascular fraction cells, (b) centrifuging said stromal vascular fraction cells, and (c) resuspending said stromal vascular fraction cells in 10% DMSO/90% FBS solution, optionally at least about 10⁴ stromal vascular fraction cells per mL.

### Methods of Use—Cosmetic, Therapeutic, Research/Diagnostic

The stromal vascular fractions and cells derived therefrom, optionally mesenchymal stem cells, which are produced according to the invention have numerous applications including use in reconstructive and aesthetic plastic surgery, and therapies, especially indications wherein stem cells and differentiated cells derived therefrom have clinical or aesthetic efficacy. Because the subject methods avoid the use of collagenase or other substances which are undesired for infusion in humans the subject vascular fractions and cells contained therein may be directly infused into patients in need thereof. The patient may be autologous, i.e., derived from the same donor or the cells may be infused into a compatible donor. Methods of HLA tissue matching cells for infusion into patients are well known in the art. Accordingly, a recipient patient may be matched using HLA tissue matching with a donor patient (e.g., the donor tissue is harvested, subjected to ultrasonic cavitation, the SVF is isolated, optionally further processed, and then administered to an HLA-matched recipient.)

For example, major histo-compatibility complex or HLA antigens may be used for matching between a SVF donor and a SVF recipient. In this process, it is analyzed which six of the HLA antigens both individuals have and a determination of the closeness of tissue matching is made. A six-antigen match (both people have the same set of six antigens) is the best compatibility between a donor recipient pair who is not identical twins. This match occurs 25 percent of the time between siblings having the same mother and father and also occurs from time-to-time in the general population. Thus, the SVF isolated using the methods disclosed herein may be used in allogeneic matches for transplantation therapies.

Further, the stromal vascular fraction cells, optionally mesenchymal stem cells derived therefrom, unexpectedly maintain hematopoietic factors contributing to their effectiveness in therapeutic uses, methods, and compositions.

The ultrasonic cavitation methods described herein mechanically treat tissue ex vivo in the absence of collagenase to lyse fat cells and the blood cells contained therein and the resultant sonically treated composition (from which the fat is removed) is then used to obtain a stromal vascular fraction which can be infused directly in patients in need thereof or it can be further processed to purify (and expand in culture if desired) desired cell types such as mesenchymal stem cells, endothelial cells, and other cells found in tissue.

The SVF cells isolated by the methods described herein may be used in patients such as for tissue reconstruction, tissue regeneration, wound healing, breast augmentation or reconstruction, in tissue fillers for plumping areas that have lost fullness, such as via aging or because of disease such as the face, lips, the buttocks, and similar areas where such an effect is desired. In particular, contemplated uses of the SVF cells isolated using the methods described herein include use with or in lieu of tissue fillers, e.g., for treating gum recession, loss of bone, including e.g., the jaw. Further, the SVF cells isolated by the methods described herein may be used in methods of treating orthopedic problems, arthritis, migraine, multiple sclerosis, autism, diabetes, optionally Type I Diabetes, wounds, ulcers, ischemic heart failure, rheumatoid arthritis, post-infarct remodeling, chronic obstructive pulmonary disease (COPD), plantar fascitis, rotator cuff injuries, and tennis elbow.

The isolated mesenchymal stem cells or other cells are derived from the stromal vascular fraction isolated using the methods described herein may be used to promote wound healing, breast augmentation, breast reconstruction, tissue engineering, treatment of ulcers in the gastrointestinal tract, or other applications.

After ultrasonic cavitation the isolated mesenchymal stem cells, optionally other cells derived therefrom, may be infused or administered into a patient for a specific cosmetic or therapeutic procedure. For example, mesenchymal stem cells may be isolated from the stromal vascular faction cells isolated using the methods described herein. The inventor surprisingly found that the stromal vascular faction cells isolated using the methods described herein produce a higher yield of mesenchymal stem cells as compared to prior art methods.

Somatic tissue stem cells can be isolated from the subject stromal vascular fraction by fractionation using fluorescence activated call sorting (FACS) with unique cell surface antigens to isolate specific subtypes of stem cells (such as adipose derived stem cells) for injection into recipients following expansion in vitro, as described herein.

The donor may be the same patient who is to be treated with the stromal or mesenchymal stem cells derived therefrom (e.g., autograft). Additionally, the donor may be an allogeneic donor that is immune compatible with the treated individual. For example, a patient may undergo liposuction to obtain the tissue. The tissue may be subjected to the methods described herein to isolate the stromal vascular fraction (SVF), optionally mesenchymal stem cells derived therefrom. The stromal vascular fraction (SVF), optionally mesenchymal stem cells derived therefrom, may then be used in therapeutic methods, uses, or compositions.

Cells may be derived from the individual to be treated or a matched donor. Those having ordinary skill in the art can readily identify matched donors using standard techniques and criteria. For example, the stromal vascular fraction cells are vascular and reside in the walls of all blood vessels in the body and now that the method described herein can be used to harvest these cells from non-living tissue. The harvested cells can be stored in cryostorage based on their donor's blood type and be used allogenically with culturing. For example, the SVF may be stored and be readily available to treat patients for a multitude of injuries (e.g., orthopedic, post myocardiacal infarction). The stromal vascular fraction cells stored may be matched to the patient by blood type and/or tissue type to ensure an allogeneic match. The stromal vascular fraction cells may be stored at +4° C., −20° C., or −70° C.

The SVF cells isolated by the method described herein may be used in autograft methods. For example, a patient may have tissue removed, optionally adipose tissue (e.g., by liposuction), the SVF cells may be isolated from the tissue, optionally adipose tissue, using the methods described herein, and then transplanted to another area of the same patient.

The SVF cells isolated by the methods described herein may be used in allogeneic transplant methods. For example, a first patient may have tissue removed, optionally adipose tissue (e.g., by liposuction), the SVF cells may be isolated from the tissue, optionally adipose tissue, using the methods described herein, and then transplanted to a second allogeneic patient.

The SVF cells isolated by the methods described herein may be used in allogeneic transplant methods. For example, a first patient may have tissue removed (e.g., by liposuction), the SVF cells may be isolated from the tissue using the methods described herein, and then transplanted to a second allogeneic patient. For example, the SVF cells isolated by the methods described herein may be used in methods for treating osteoarthritis, sports medicine injuries, including but not limited to tears and sprains of the ligaments and tendons, gingival gum regeneration, dermal treatment of burns and non-healing wounds, hair loss in men and women, rheumatoid arthritis, multiple sclerosis, ALS disease, autism, tinnitus, and bone fractures.

The SVF cells isolated by the methods described herein may be used in xenotransplant methods. For example, adipose tissue removed (e.g., by liposuction) from an animal donor, the SVF cells may be isolated from the adipose tissue using the methods described herein, and then transplanted into a human patient. Adipose tissue removed (e.g., by liposuction) from an animal donor, optionally a pig, the SVF cells may be isolated from the adipose tissue using the methods described herein, and then transplanted into a human patient to treat a disease or accelerate tissue healing. The SVF cells isolated by the methods described herein may be used to grow tissue and/organs for transplant, e.g., bone, Islets of Langerhans, mitral valve. For example, methods for treating osteoarthritis, sports medicine injuries, including but not limited to tears and sprains of the ligaments and tendons, gingival gum regeneration, dermal treatment of burns and non-healing wounds, hair loss in men and women, rheumatoid arthritis, multiple sclerosis, ALS disease, autism, tinnitus, and bone fractures.

For example the vascular cell fractions may be administered alone or in combination with tissue fillers (e.g., Juvederm) or scaffolds or matrices used to promote tissue regeneration or reconstruction, e.g., breast or other cancer reconstructive surgeries, foot surgery, breast augmentation, penile implants, facial fillers, joint or cartilage surgery, neck surgery, and the like. Injectable dermal fillers provide a noninvasive option for providing a scaffold for SVF cells in therapeutic methods, uses, and compositions. The subject vascular cell fractions and stem cells derived therefrom may be used in cosmetic compositions used for topical application to the skin to effect rejuvenation and promote radiance, and reduce wrinkling, optionally in combination with a dermal filler. For example, collagen, Autologen® (autologous collagen dispersion), Isolagen® (autologous fibroblast composition), Dermalogen® (injectable human dermal implant material), hyaluronic acid, calcium hydroxyapatite, and/or synthetic poly-lactic acid may be used as a scaffold for SVF cells in therapeutic methods, uses, and compositions. Additionally, a dermal filler composition comprising an acrylate/methacrylate copolymer may be used as scaffold for SVF cells in therapeutic methods, uses, and compositions. See U.S. Pat. No. 7,910,134. Another permanent microsphere-based injectable dermal filler contains larger non-resorbable microspheres made of polymethyl methacrylate (PMMA), each having a diameter of between 30 and 42 μm and a smooth surface, and a highly purified bovine collagen gel in a ratio of 20% PMMA to 80% bovine collagen.

The SVF compositions described herein may be used in the manufacture of cosmetic surgery products, optionally comprising dermal fillers. For example, a method for cosmetic surgery may comprise administering the SVF compositions described herein. A composition for cosmetic surgery may comprise an effective amount of the SVF compositions described herein. The SVF compositions described herein may be used in the manufacture of cosmetic surgery products, optionally comprising dermal fillers, for the reduction of a skin defect.

A method for reducing a skin defect may comprise administering the SVF compositions described herein. A composition for reduction of a skin defect may comprise an effective amount of the SVF compositions described herein. The SVF compositions described herein may be used in the manufacture of cosmetic surgery products, optionally comprising dermal fillers, for reducing wrinkles, improving rosacea, increase skin thickness, increase skin tone, improve skin texture, and tighten skin.

A method for reducing wrinkles, improving rosacea, increase skin thickness, increase skin tone, improve skin texture, and tighten skin may comprise administering the SVF compositions described herein.

A composition for reducing wrinkles, improving rosacea, increase skin thickness, increase skin tone, improve skin texture, and tighten skin may comprise an effective amount of the SVF compositions described herein. In another embodiment, the use, method, or composition may be for the reduction of a skin defect, the skin defect may be a dynamic wrinkle, a fine wrinkles or a static wrinkle. In another embodiment, the dynamic wrinkle may be a forehead crease, a brow burrow or an eye line (crow's feet). In another embodiment, the static wrinkle may be a skin fold wrinkle resulting from sagging skin. In another embodiment, the skin defect may be a medical condition selected from the group consisting of an acne scar, optionally a “rolling” scar, a “boxcar” scar or an “ice pick” scar, a surgical scar, trauma scar, a large pore and a soft tissue contour defect. In another embodiment, the wrinkle or scar may be the result of loss of collagen and hyaluronic acid in the skin during the aging process. In another embodiment, the wrinkle or scar may be the result of premature aging, optionally premature aging caused by overexposure to sunlight, overexposure to environmental pollutants, smoking tobacco products, exposure to cigarette smoke, poor nutrition, and skin disorders.

The SVF compositions described herein may be used in methods of treating rosacea, psoriasis, acne, eczema, and atopic dermatitis, optionally the SVF composition may be applied topically. Further, the SVF compositions described herein may be used in cosmetic methods treat wrinkles, tone, text, large pores, dullness, or loose skin, optionally the SVF composition may be applied topically.

The compositions comprising SVF may be used in a method of augmenting soft tissue to provide of a skin defect. The compositions comprising SVF may be used in a method of augmenting soft tissue to provide reduction of a skin defect comprising topically applying to the skin defect a composition comprising SVF.

The compositions comprising SVF may be used in may be applied topically to the skin defect in a method of augmenting soft tissue to provide reduction of a skin defect. The skin defect may be a result of loss of collagen and hyaluronic acid in the skin during the aging process. The skin defect may be a result of premature aging, said premature aging caused by overexposure to sunlight, overexposure to environmental pollutants, smoking tobacco products, exposure to cigarette smoke, poor nutrition, and skin disorders.

The compositions comprising SVF may be used in a method of augmenting soft tissue to provide reduction of a skin defect, the skin defect may be a dynamic wrinkle, a fine wrinkles or a static wrinkle. The dynamic wrinkle may be a forehead crease, a brow burrow or an eye line (crow's feet). The static wrinkle may be a skin fold wrinkle resulting from sagging skin. The skin defect is a medical condition selected from the group consisting of an acne scar, for example, a “rolling” scar, a “boxcar” scar or an “ice pick” scar, a surgical scar, trauma scar, a large pore and a soft tissue contour defect. The medical condition may be a deformity that requires re-contouring, such as a small tissue defect (e.g., after animal bite(s)) or a deformity related to trauma where the deformity is cosmetically unappealing. The augmentation may be after plastic surgery to achieve symmetry or a desired result.

The method of augmenting soft tissue to provide long-term reduction of a skin defect, a “long-term” reduction of a skin defect is of a duration of at least one year. A long-term reduction of a skin defect is of a duration of from at least one year to about five years. A long-term reduction of a skin defect is of a duration from about five years to about ten years. A long-term reduction of a skin defect is of a duration from about ten years or longer.

The stromal vascular fraction produced according to the invention may be purified into desired cell types, e.g., a pure population of mesenchymal stem cells, endothelial precursor cells, hematopoietic stem cells, and these cells propagated in vitro using cell culture methods well known to those skilled in the art. As discussed herein those skilled in the art conventionally separate stem cells from other cells by FACS and other cell sorting methods based on the expression of characteristic markers.

The resultant purified stem cells may be injected into desired organs to effect tissue repair, e.g. into heart muscle to effect repair of the heart muscle, after a heart attack, into brain or spinal fluid to effect neural or nerve regeneration, such as Parkinson's or Alzheimer's patients, into the bone or cartilage of individuals in need thereof such as individuals suffering from age, exertion, or disease related bone or cartilage loss. For example, isolated stem cells derived from SVF cells may be used in autograft procedures by injection into desired organs to effect tissue repair, e.g. into heart muscle to effect repair of the heart muscle, after a heart attack, into brain or spinal fluid to effect neural or nerve regeneration, such as Parkinson's or Alzheimer's patients, into the bone or cartilage of individuals in need thereof such as individuals suffering from age, exertion, or disease related bone or cartilage loss. Also, the isolated stem cells derived from SVF cells may be used in these procedures may be from an allogeneic donor or as a xenotransplantation. Further, the SVF cells may be formulated in compositions for allogeneic transplant. The SVF cells may be formulated in compositions for autograft transplant. The SVF cells may be formulated in compositions for xenotransplant.

These purified stem cells may alternatively be cultured under conditions that give rise to desired cell lineages. For example mesenchymal and stromal stem cells comprised in the subject fraction s may be differentiated into desired cell types including fibroblasts, neural cells, hematopoietic cells, myocytes, chondrocytes, and other cell types. In addition these cell types, e.g., fibroblast populations may be seeded on a scaffold, which may be used in wound healing.

An automated system for separating and concentrating clinically safe regenerative cells from adipose tissue that are suitable for re-infusion into a subject may be used in conjunction with the ultrasonic cavitation methods described herein. A system for separating and concentrating cells from tissue in accordance with the invention may include one or more of a collection chamber, a processing chamber, a waste chamber, an output chamber and a sample chamber. The various chambers are coupled together via one or more conduits such that fluids containing biological material may pass from one chamber to another in a closed, or functionally closed, sterile fluid/tissue pathway which minimizes exposure of tissue, cells, biologic and non-biologic materials with contaminants. The waste chamber, the output chamber and the sample chamber are optional. The system contains clinically irrelevant quantities of endotoxin. The system also includes a plurality of filters. The filters are effective to separate the stem cells and/or progenitor cells from, among other things, collagen, free lipids, adipocyte, that may be present in the solution after ultrasonication cavitation of the tissue sample. The filter assembly may include a hollow fiber filtration device. A filter assembly includes a percolative filtration device, which may or may not be used with a sedimentation process. The filter assembly may comprise a centrifugation device, which may or may not be used with an elutriation device and process. The system may comprise a combination of these filtering devices. The filtration functions can be two-fold, with some filters removing things from the final concentration such as collagen, free lipid, free adipocytes, and with other filters being used to concentrate the final product.

One or more components of the system are automated and include an internal processing device and associated software programs which control many of the processing functions. Components of the system may be disposable, such that portions of the system can be disposed of after a single use. Such a system also comprises a re-usable component which includes the processing device (computer and associated software programs) and other components such as motors, pumps.

A method of treating a patient may comprise (a) providing a tissue removal system; (b) removing tissue from a patient using the tissue removal system, the tissue having a concentration of stem cells; (c) processing at least a part of the tissue by use of ultrasonic sonication for a time sufficient to explode all or most of the fat cells and release the stromal vascular cells into a suitable fluid medium, e.g. phosphate buffered saline solution, (d) allowing the treated solution to settle such that the fat rises to the top of the solution and the fat is removed in order to obtain a concentrated stromal vascular fraction containing regenerative cells (e.g., mesenchymal stem cells, endothelial precursor cells, hematopoietic stem cells) higher than the concentration of regenerative cells of the tissue before processing, wherein the processing occurs within a sterile, closed or functionally closed system; and (e) administering the concentrated regenerative cells to a patient, to thereby treat the patient.

The stromal vascular fraction cells, optionally mesenchymal stem cells, endothelial precursor cells, or hematopoietic stem cells, may be administered directly into the patient. In other words, the cells (e.g., the stem cells and/or endothelial precursor cells contained in the stromal vascular fraction) may be administered to the patient without being removed from the system or exposed to the external environment of the system before being administered to the patient. Providing a closed system reduces the possibility of contamination of the material being administered to the patient. Thus, processing the tissue in a closed system provides advantages because the cell population is more likely to be sterile. The only time the stem cells and/or endothelial precursor cells are exposed to the external environment, or removed from the system, is when the cells are being withdrawn into an application device and being administered to the patient. The application device can also be part of the closed system. Thus, the cells used may not processed for culturing or cryopreserved.

The cells derived from the stromal vascular fraction, optionally mesenchymal stem cells, endothelial precursor cells, or hematopoietic stem cells, that have been concentrated, as described above, may be administered to a patient without further processing, or may be administered to a patient after being mixed with other tissues or cells. The concentrated cells (e.g., mesenchymal stem cells, endothelial precursor cells, or hematopoietic stem cells) are mixed with one or more units of tissue that has not been similarly processed. Thus, by practicing the methods of the invention, a composition comprising tissue with an enhanced concentration of cells derived from the stromal vascular fraction, optionally mesenchymal stem cells, endothelial precursor cells, or hematopoietic stem cells, may be administered to the patient. The volumes of the various units of tissue may be different. For example, one volume may be at least 25% greater than the volume of another unit of tissue. Furthermore, one volume may be at least about 50%, at least about 100%, and even about 150% or more greater than the volume of another unit of tissue. In addition, the desired composition may be obtained by mixing a first unit of tissue with the concentrated cell population, which may be a cell pellet containing the desired cells from the stromal vascular fraction, optionally mesenchymal stem cells, endothelial precursor cells, or hematopoietic stem cells, with one or more other units of tissue. These other units will not have an increased concentration of stem cells, optionally mesenchymal stem cells, endothelial precursor cells, or hematopoietic stem cells, or in other words, will have an cell concentration less than that contained in the first unit of tissue. One of the units is cryopreserved material that contains, for example, an increased concentration of stem cells, optionally mesenchymal stem cells, endothelial precursor cells, or hematopoietic stem cells.

At least a portion of the desired cell population derived from the stromal vascular fraction may be stored for later implantation/infusion. The population may be divided into more than one aliquot or unit such that part of the population of stem cells may be retained for later application while part is applied immediately to the patient. Moderate to long-term storage of all or part of the cells in a cell bank is also within the scope of this invention. The cells may be mixed with one or more units of fresh or preserved tissue to provide a composition containing the stem cells at a higher concentration than a unit of tissue prior to processing.

At the end of processing, the concentrated cells, optionally mesenchymal stem cells, endothelial precursor cells, or hematopoietic stem cells, may be loaded into a delivery device, such as a syringe, for placement into the recipient by either subcutaneous, intravenous, intramuscular, or intraperitoneal techniques. In other words, cells may be placed into the patient by any means known to persons of ordinary skill in the art, for example, they may be injected into blood vessels for systemic or local delivery, into tissue (e.g., cardiac muscle, or skeletal muscle), into the dermis (subcutaneous), into tissue space (e.g., pericardium or peritoneum), or into tissues (e.g., periurethral emplacement), or other location, such as placement by needle or catheter, or by direct surgical implantation in association with additives such as a preformed matrix.

The cell population, optionally mesenchymal stem cells, endothelial precursor cells, or hematopoietic stem cells, may be applied alone or in combination with other cells, tissue, tissue fragments, demineralized bone, growth factors (e.g., insulin or drugs, e.g., members of the thiaglitazone family), biologically active compounds, biologically inert compounds, resorbable plastic scaffolds, or other additive intended to enhance the delivery, efficacy, tolerability, or function of the population. The cell population, optionally mesenchymal stem cells, endothelial precursor cells, or hematopoietic stem cells, may also be modified by insertion of DNA or by placement in cell culture in such a way as to change, enhance, or supplement the function of the cells for derivation of a cosmetic, structural, or therapeutic purpose. For example, gene transfer techniques for stem cells are known by persons of ordinary skill in the art, as disclosed in Mosca, et al. (2000) Clin Orthop (379 Suppl): S71-90, and may include viral transfection techniques, and more specifically, adeno-associated virus gene transfer techniques, as disclosed in Walther and Stein (2000) Drugs 60(2): 249-71, and Athanasopoulos, et al. (2000) Int J Mol Med 6(4): 363-75. Non-viral based techniques may also be performed as disclosed in Muramatsu, et al. (1998) Int J Mol Med 1(1): 55-62.

The cells, optionally mesenchymal stem cells, endothelial precursor cells, or hematopoietic stem cells, may be mixed with unprocessed fragments of tissue and placed back into the recipient using a very large gauge needle or liposuction cannula. Transfer of autologous fat without supplementation with processed cells is a common procedure in plastic and reconstructive surgery. Cells derived from the stromal vascular fraction obtained by the methods described herein are, for example, substantially depleted of mature adipocytes may provide an environment that supports prolonged survival and function of the graft.

The cells, optionally mesenchymal stem cells, endothelial precursor cells, or hematopoietic stem cells, may be placed into the recipient and surrounded by a resorbable plastic sheath such as that manufactured by MacroPore Biosurgery, Inc. U.S. Pat. Nos. 6,269,716 and 5,919,234. In this setting the sheath would prevent prolapse of muscle and other soft tissue into the area of a bone fracture thereby allowing the emplaced processed tissue-derived cells to promote repair of the fracture. The beneficial effect may be enhanced by supplementation with additional components such as pro-osteogenic protein growth factors, biological scaffolds, or artificial scaffolds.

The cells, optionally mesenchymal stem cells, endothelial precursor cells, or hematopoietic stem cells, may be combined with a gene encoding a pro-osteogenic growth factor which would allow cells to act as their own source of growth factor during bone healing or fusion. Addition of the gene could be by any technology known in the art including but not limited to adenoviral transduction, “gene guns,” liposome-mediated transduction, and retrovirus or Lentivirus-mediated transduction.

Particularly when the cells and/or tissue containing the cells are administered to a patient other than the patient from which the cells and/or tissue were obtained, one or more immunosuppressive agents may be administered to the patient receiving the cells and/or tissue to reduce, and preferably prevent, rejection of the transplant. Examples of immunosuppressive agents suitable with the methods disclosed herein include agents that inhibit T-cell/B-cell costimulation pathways, such as agents that interfere with the coupling of T-cells and B-cells via the CTLA4 and B7 pathways, as disclosed in U.S. Patent Application Publication No. 2002/0182211. Other examples include but are not limited to cyclosporin, myophenylate mofetil, rapamicin, and anti-thymocyte globulin.

The cells, optionally mesenchymal stem cells, endothelial precursor cells, or hematopoietic stem cells, may be administered to a patient with one or more cellular differentiation agents, such as cytokines and growth factors. Examples of various cell differentiation agents are disclosed in Gimble, et al. (1995) J Cell Biochem 58(3): 393-402; Lennon, et al. (1995) Exp Cell Res 219(1): 211-22; Majumdar, et al. (1998) J Cell Physiol 176(1): 57-66; Caplan and Goldberg (1999) Clin Orthop (367 Suppl): 512-6; Ohgushi and Caplan (1999) J Biomed Mater Res 48(6): 913-27; Pittenger, et al. (1999) Science 284(5411): 143-7; Caplan and Bruder (2001) Trends Mol Med 7(6): 259-64; Fukuda (2001) Artif Organs 25(3): 187-93; Worster, et al. (2001) J Orthop Res 19(4): 738-49; Zuk, et al. (2001) Tissue Eng 7(2): 211-28; and Mizuno, et al. (2002) Plast Reconstr Surg 109(1): 199-209.

By administering the cells, optionally mesenchymal stem cells, endothelial precursor cells, or hematopoietic stem cells, to a patient, one can treat numerous diseases, including, and not limited to, bone-related disorders, diseases, or injuries, including slow/non-union fractures, osteoporosis (age-related or chemotherapy-induced), inherited diseases of bone (osteogenesis imperfecta); adipose related disorders or diseases; liver related diseases, disorders, or injuries, including liver failure, hepatitis B, and hepatitis C; myocardial infarctions, including heart attack or chronic heart failures; renal diseases or kidney damage; retinal diseases or damage or necrosis; wound healing (e.g., from surgery or diabetic ulcers); skeletal muscle disorders both traumatic and inherited; cartilage and joint repair both traumatic and autoimmune; lung injuries; diabetes; intestinal disorders; nervous system disorders, diseases, or injuries, such as central nervous systems disorders, diseases, or injuries, including spinal cord injuries, Parkinson's disease, Alzheimer's disease, and stroke.

Further, the cells, optionally mesenchymal stem cells, endothelial precursor cells, or hematopoietic stem cells, to a patient, one can treat numerous diseases, including, and not limited to, osteoarthritis, sports medicine injuries, including but not limited to tears and sprains of the ligaments and tendons, gingival gum regeneration, dermal treatment of burns and non-healing wounds, hair loss in men and women, rheumatoid arthritis, multiple sclerosis, ALS disease, autism, tinnitus, and bone fractures.

The cells, optionally mesenchymal stem cells, endothelial precursor cells, or hematopoietic stem cells, to a patient, may be formulated into compositions for the treatment of diseases including but not limited to bone-related disorders, diseases, or injuries, including slow/non-union fractures, osteoporosis (age-related or chemotherapy-induced), inherited diseases of bone (osteogenesis imperfecta); adipose related disorders or diseases; liver related diseases, disorders, or injuries, including liver failure, hepatitis B, and hepatitis C; myocardial infarctions, including heart attack or chronic heart failures; renal diseases or kidney damage; retinal diseases or damage or necrosis; wound healing (e.g., from surgery or diabetic ulcers); skeletal muscle disorders both traumatic and inherited; cartilage and joint repair both traumatic and autoimmune; lung injuries; diabetes; intestinal disorders; nervous system disorders, diseases, or injuries, such as central nervous systems disorders, diseases, or injuries, including spinal cord injuries, Parkinson's disease, Alzheimer's disease, and stroke.

Additionally, the stromal vascular faction cells may be used to formulate compositions for the treatment of loss of bone, optionally the jaw, amyotrophic lateral sclerosis (ALS), arthritis, optionally rheumatoid arthritis, autism, diabetes, optionally Type I diabetes, bone fractures, chronic obstructive pulmonary disease (COPD), dermal treatment for burns and non-healing wounds, enterocutaneous fistula (HULPUTC), gingival gum regeneration, hair loss (in both men and women), gum recession, ischemic heart failure, microvascular protection treatment in a myocardial infarction, migraine, multiple sclerosis, orthopedic problems, osteoarthritis, plantar fascitis, recto-vaginal fistula, rheumatoid arthritis, rotator cuff injuries, sports medicine injuries, optionally tears and sprains of the ligaments and tendons, tennis elbow, tinnitus, ulcers, or wounds. Further, the cells, optionally mesenchymal stem cells, endothelial precursor cells, or hematopoietic stem cells, may be used to formulate compositions for the treatment of numerous diseases, including, and not limited to, osteoarthritis, sports medicine injuries, including but not limited to tears and sprains of the ligaments and tendons, gingival gum regeneration, dermal treatment of burns and non-healing wounds, hair loss in men and women, rheumatoid arthritis, multiple sclerosis, ALS disease, autism, tinnitus, and bone fractures.

Also, the stromal vascular fraction cells may be used in methods of treatment of the loss of bone, optionally the jaw, amyotrophic lateral sclerosis (ALS), arthritis, optionally rheumatoid arthritis, autism, diabetes, optionally Type I diabetes, bone fractures, chronic obstructive pulmonary disease (COPD), dermal treatment for burns and non-healing wounds, enterocutaneous fistula (HULPUTC), gingival gum regeneration, hair loss (in both men and women), gum recession, ischemic heart failure, microvascular protection treatment in a myocardial infarction, migraine, multiple sclerosis, orthopedic problems, osteoarthritis, plantar fascitis, recto-vaginal fistula, rheumatoid arthritis, rotator cuff injuries, sports medicine injuries, optionally tears and sprains of the ligaments and tendons, tennis elbow, tinnitus, ulcers, or wounds.

The stromal vascular fraction cells may be formulated into a composition comprising an additional pharmaceutical or agent, or alternatively a polynucleotide that encodes for a therapeutic agent or for an inhibiting nucleic acid. Examples of nuclear acids include, a ribozyme, an antisense oligonucleotide, a double stranded RNA, a double-stranded interfering RNA (iRNA), a triplex RNA, an RNA aptamer, and at least a portion of an antibody molecule that binds to the gene product and inhibits its activity.

The stromal vascular fraction obtained by the ultrasonic cavitation methods described herein may be stored in liquid nitrogen (i.e., −70° C.) or refrigerated (i.e., 4° C.). For example, one may harvest tissue from cadaveric donors less than 24 hours post-mortem. SVF cells will be isolated from the cadaveric tissue and processed using the standard ultrasonic cavitation method. The SVF cells will then be sorted/graded with immuno-affinity columns to isolate/remove allogeneic antigens that would cause a transplant reaction. The cells would be tested to match common blood types and Rh factors to recipients and then cryo-preserved and frozen to be stored for use by patients requiring cellular therapies in physician's offices and hospitals at future dates.

The subject adipose derived stromal vascular fraction or stem and endothelial precursor cells purified or derived therefrom may be induced to differentiate. In particular the following usages of cells according to the invention as described in the patent references discussed below are contemplated.

The cells obtained from the methods described herein may be used in conformable tissue implant for use in repairing or augmenting a tissue defect or injury site that may contain stem cells. The tissue implant contains a tissue carrier matrix comprising a plurality of biocompatible, bioresorbable granules and at least one tissue fragment in association with the granules. U.S. Pat. No. 7,875,296.

The cells obtained from the methods described herein may be used for repairing a damaged urinary tract tissue of a subject. U.S. Pat. No. 7,875,276.

The cells obtained from the methods described herein may be used tissue scaffolds suitable for use in repair and/or regeneration of musculoskeletal tissue when implanted in a body. U.S. Pat. No. 7,625,581.

The cells obtained from the methods described herein may be used as a tissue repair implant comprising: a tissue carrier matrix comprising a plurality of biocompatible, bioresorbable granules and at least one tissue fragment in association with the tissue carrier matrix, the at least one tissue fragment having an effective amount of viable cells that can migrate out of the tissue fragment and populate the tissue carrier matrix, wherein the tissue carrier matrix is in the form of an injectable suspension, and wherein an average maximum outer diameter of the granules is in a range of about 150 to about 600 μm. U.S. Pat. No. 7,316,822.

The cells obtained from the methods described herein may be used in a method of implanting stem or endothelial precursor cells into a body of a patient, said method comprising the steps of: providing a support structure, harvesting a polysaccharide-based modified biofilm from bacteria, attaching viable cells for implantation to the support structure with the polysaccharide-based modified biofilm, and connecting one portion of a blood vessel in the patient's body with a first portion of the support structure, and connecting another portion of a blood vessel in the patient's body with a second portion of the support structure. U.S. Pat. No. 7,299,805.

The cells obtained from the methods described herein may be used in an implantable biodegradable device containing a fibrous matrix, the fibrous matrix being constructed from fibers A and fibers B, wherein fibers A biodegrade faster than fibers B, fibers A and fibers B are present in relative amounts and are organized such that the fibrous matrix is provided with properties useful in repair and/or regeneration of mammalian tissue, and which may contain mesenchymal or stromal stem or endothelial precursor cells. U.S. Pat. No. 7,192,604.

The cells obtained from the methods described herein may be induced to express at least one phenotypic characteristic of a neuronal, astroglial, hematopoietic progenitor, or hepatic cell and then used in therapy or tissue reconstruction. U.S. Pat. No. 7,078,230.

The cells obtained from the methods described herein may be used in methods and compositions for directing adipose-derived stromal cells cultivated in vitro to differentiate into cells of the chondrocyte lineage. The cells obtained from the methods described herein may be used therapeutic treatment of a number of human conditions and diseases including repair of cartilage in vivo. U.S. Pat. Nos. 7,033,587, 6,841,150, and 6,429,013.

The cells obtained from the methods described herein may be used in methods of making bioremodelable graft prostheses prepared from cleaned tissue material derived from animal sources. The bioengineered graft prostheses of the invention are prepared using methods that preserve cell compatibility, strength, and bioremodelability of the processed tissue matrix. The bioengineered graft prostheses are used for implantation, repair, or use in a mammalian host. These prostheses may contain mesenchymal stem or endothelial precursor cells. U.S. Pat. No. 6,986,735.

In particular the invention provides a lipo-derived stem cell substantially free of adipocytes and include treatment of use with or in lieu of tissue fillers, as a gum recession, loss of bone, including the jaw, treatment of orthopedic problems, treatment of arthritis, treatment of migraine, treatment of multiple sclerosis, treatment of autism, treatment of diabetes, treatment of wounds, treatment of ulcers, treatment of COPD, treatment of plantar fascitis, treatment of rotator cuff, and treatment of tennis elbow.

The cells obtained from the methods described herein may be used for treatment of a disease, including transplantation, in combination with a biocompatible polymer.

Biocompatible polymers, for example, include but are not limited to, homopolymers, co-polymers, block polymers, cross-linkable or crosslinked polymers, photoinitiated polymers, chemically initiated polymers, biodegradable polymers, and nonbiodegradable polymers. In some embodiments, the SVF cell transplant may comprise a polymer matrix that is nonpolymerized, to allow it to be combined with a tissue, organ, or engineered tissue in a liquid or semi-liquid state, for example, by injection. In other embodiments, the SVF cell transplant comprising liquid matrix may polymerize or substantially polymerize in situ. In still other embodiments, the SVF cell transplant may be admixed with the polymer, polymerized or substantially polymerized prior to injection. Such injectable compositions are prepared using conventional materials and methods know in the art. See, e.g., Knapp, et al. (1977) Plastic and Reconstr. Surg. 60:389-405; Fagien (2000) Plastic and Reconstr. Surg. 105: 362-73 and 2526-28; Klein, et al. (1984) J. Dermatol. Surg. Oncol. 10: 519-22; Klein (1983) J. Amer. Acad. Dermatol. 9: 224-28; Watson, et al. (1983) Cutis 31: 543-46; Klein (2001) Dermatol. Clin. 19: 491-508; Klein (1999) Pedriat. Dent. 21: 449-50; Skorman (1987) J. Foot Surg. 26: 5115; Burgess (1992) Facial Plast. Surg. 8: 176-82; Laude, et al. (2000) J. Biomech. Eng. 122: 231-35; Frey, et al. (1995) J. Urol. 154: 812-15; Rosenblatt, et al. (1994) Biomaterials 15: 985-95; Griffey, et al. (2001) J. Biomed. Mater. Res. 58: 10-15; Stenburg, et al. (1999) Scfand. J. Urol. Nephroi. 33: 355-61; Sclafani, et al. (2000) Facial Plast. Surg. 16: 29-34; Spira, et al. (1993) Clin. Plast. Surg. 20: 18188; Ellis, et al. (2001) Facila Plast. Surg. Clin. North Amer. 9: 405-11; Alster, et al. (2000) Plastic Reconstr. Surg. 105: 2515-28; and U.S. Pat. Nos. 3,949,073 and 5,709,854.

The polymerized or nonpolymerized matrix may comprise collagen, including but not limited to contracted and non-contracted collagen gels, hydrogels comprising, for example, but not limited to, fibrin, alginate, agarose, gelatin, hyaluronate, polyethylene glycol (PEG), dextrans, including dextrans that are suitable for chemical crosslinking, photocrosslinking, or both, albumin, polyacrylamide, polyglycolyic acid, polyvinyl chloride, polyvinyl alcohol, poly(n-vinyl-2-pyrollidone), poly(2-hydroxy ethyl methacrylate), hydrophilic polyurethanes, acrylic derivatives, or pluronics (e.g., polypropylene oxide and polyethylene oxide copolymer.)

The fibrin or collagen is autologous or allogeneic with respect to the intended recipient. The skilled artisan will appreciate that the matrix may comprise non-degradable materials, for example, but not limited to, expanded polytetrafluoroethylene (ePTFE), polytetrafluoroethylene (PTFE), polyethyleneterephthalate (PET), polyurethane, polyethylene, polycabonate, polystyrene, silicone, or selectively degradable materials (e.g., poly (lactic-co-glycolic acid; PLGA), PLA, or PGA). See also, Middleton, et al. (2000) Biomaterials 21: 2335-2346; Middleton, et al. Medical Plastics and Biomaterials (March/April 1998) pages 30-37; Handbook of Biodegradable Polymers, Domb, Kost, and Domb, Eds., 1997, Harwood Academic Publishers, Australia; Rogalla (1997) Minim. Invasive Surg. Nurs. 11: 67-69; Klein (2001) Facial Plast. Surg. Clin. North Amer. 9: 205-18; Klein, et al. (1985) J. Dermatol. Surg. Oncol. 11: 337-39; Frey, et al. (1995) J. Urol. 154: 812-15; Peters, et al. (1998) J. Biomed. Mater. Res. 43: 422-27; and Kuijpers, et al. (2000) J. Biomed. Mater. Res. 51: 136-45.

The present invention provides for a method of providing a stromal vascular fraction composition to a clinical site comprising (a) thawing vials of cryopreserved stromal vascular fraction cells, (b) resuspending the stromal vascular fraction cells in media, (c) centrifuging the stromal vascular fraction cells, (d) resuspending the stromal vascular fraction cells in media, (e) aliqouting the stromal vascular fraction cells into vials, and (f) transferring to the clinical site. In one embodiment, the resuspension and centrifugation steps may be repeated at least 1, 2, 3, 4, or 5 times. In another embodiment, the stromal vascular fraction product is transported to the clinical site within at least about 1, 2, 3, 4, 5, 6, 7, 8, 9, or 10 hours of completion of step (e). In a further embodiment, the vials may be labeled.

The present invention also provides a method for a providing stromal vascular fraction cell preparation for sale comprising (a) producing stromal vascular fraction cells and (b) preparing said stromal vascular fraction cell preparations for transfer to a customer. In one embodiment, the method may comprise cryopreserving the stromal vascular fraction cells. In another embodiment, the method comprises offering said stromal vascular fraction cell preparations for sale. In a further embodiment, the method comprises advertising the stromal vascular fraction cell preparations. Donor reimbursement costs may include the cost of fat harvesting in the case of cadaveric tissue donation.

Additionally, the stromal vascular fraction cells prepared by the ultrasonic cavitation methods described in U.S. Patent Application Publication No. 2012/0164113 are different than the SVF cell product produced using enzymes, e.g., collagenase, in part because the methods described herein do not wash out the extracellular matrix (ECM). The extracellular matrix (ECM) is the extracellular part of animal tissue that usually provides structural support to the animal cells in addition to performing various other important functions. The extracellular matrix is the defining feature of connective tissue in animals. Extracellular matrix includes the interstitial matrix and the basement membrane. Interstitial matrix is present between various animal cells (i.e., in the intercellular spaces). Gels of polysaccharides and fibrous proteins fill the interstitial space and act as a compression buffer against the stress placed on the ECM. Basement membranes are sheet-like depositions of ECM on which various epithelial cells rest. Due to its diverse nature and composition, the ECM can serve many functions, such as providing support, segregating tissues from one another, and regulating intercellular communication. The extracellular matrix regulates a cell's dynamic behavior. In addition, it sequesters a wide range of cellular growth factors, and acts as a local depot for them. Changes in physiological conditions can trigger protease activities that cause local release of such depots. This allows the rapid and local growth factor-mediated activation of cellular functions, without de novo synthesis. Formation of the extracellular matrix is essential for processes like growth, wound healing and fibrosis. Therefore, the stromal vascular fraction cells prepared by the ultrasonic cavitation methods described in U.S. Patent Application Publication No. 2012/0164113 comprise extracellular matrix components, unlike SVF prepared by enzymatic treatment, and thus may have better healing properties.

Bone marrow is a leading source of adult stem cells, which are increasingly used for research and therapeutic interventions, but extracting the cells is an arduous and often painful process. Accordingly, The allogeneic SVF compositions described herein may be used lieu of bone marrow for treatment of conditions treated by bone marrow transplantation. The allogeneic stromal vascular fraction described herein may be used in lieu of bone marrow as a source of adult stem cells. The allogeneic stromal vascular fraction described herein has several advantages over bone marrow including a higher yield of stem cells and greater cell viability.

The SVF may be admixed to form a composition and stored for 1-10 days or 1-6 days, optionally 10 days or 3 months.

### Cosmetic Products and Methods

A method for formulating a topical skin cream comprising isolating SVF from a patient and compounding to form a topical skin cream may comprise autologous SVF. In another embodiment, the SVF composition may be administered topically.

The SVF compositions described herein may be a serum, lotion, or cream.

The SVF compositions described herein may be used to compound a topical skin cream comprising a patient's own growth factors derived from SVF from the same patient (autologous). The SVF compositions described herein, optionally a topical skin cream, may be patient specific and compounded by the physician at the time of the patient visit.

### Xenotransplant and Veterinary Applications

The allogeneic stromal vascular fraction described herein may be used in xenotransplantation methods. For example, the allogeneic stromal vascular fraction described herein isolated from a human donor may be transplanted into an animal recipient. Further, the allogeneic stromal vascular fraction described herein may be isolated from an animal donor and transplanted into a human recipient. Additionally, the allogeneic stromal vascular fraction described herein have veterinary applications where the allogeneic stromal vascular fraction described herein may be isolated from one non-human animal and transplanted into another non-human animal.

Stem cells, optionally mesenchymal stem cells, endothelial precursor cells, or hematopoietic stem cells, may be isolated from the allogeneic stromal vascular fraction described herein. The resultant purified stem cells may be injected into desired organs to effect tissue repair, e.g. into heart muscle to effect repair of the heart muscle, after a heart attack, into brain or spinal fluid to effect neural or nerve regeneration, such as Parkinson's or Alzheimer's patients, into the bone or cartilage of individuals in need thereof such as individuals suffering from age, exertion, or disease related bone or cartilage loss. For example, isolated stem cells derived from SVF cells may be used in autograft procedures by injection into desired organs to effect tissue repair, e.g. into heart muscle to effect repair of the heart muscle, after a heart attack, into brain or spinal fluid to effect neural or nerve regeneration, such as Parkinson's or Alzheimer's patients, into the bone or cartilage of individuals in need thereof such as individuals suffering from age, exertion, or disease related bone or cartilage loss. Also, the isolated stem cells derived from the allogeneic SVF cells may be used in these procedures may be from an allogeneic donor or as a xenotransplantation. Further, the SVF cells may be formulated in compositions for allogeneic transplant. The SVF cells may be formulated in compositions for autograft transplant. The SVF cells may be formulated in compositions for xenotransplant.

The allogeneic SVF compositions described herein may be used in xenotransplant methods. For example, adipose tissue removed (e.g., by liposuction) from an animal donor, the VF cells may be isolated from the adipose tissue using the methods described herein, and then transplanted into a human patient. Adipose tissue removed (e.g., by liposuction) from an animal donor, optionally a pig, the SVF cells may be isolated from the adipose tissue using the methods described herein, and then transplanted into a human patient to treat a disease or accelerate tissue healing. The allogeneic SVF compositions described herein may be used to grow tissue and/organs for transplant, e.g., bone, Islets of Langerhans, mitral valve. For example, methods for treating osteoarthritis, sports medicine injuries, including but not limited to tears and sprains of the ligaments and tendons, gingival gum regeneration, dermal treatment of burns and non-healing wounds, hair loss in men and women, rheumatoid arthritis, multiple sclerosis, ALS disease, autism, tinnitus, and bone fractures.

The allogeneic SVF compositions described herein may be used in xenotransplant methods. For example, the SVF may be isolated from whole blood using the methods known in the art, admixed to form a SVF composition and then transplanted into a human patient. For example, SVF may be isolated from an animal donor, optionally a pig, using the methods known in the art, admixed to form a SVF composition, and then transplanted into a human patient to treat a disease or accelerate tissue healing.

All publications (e.g., Non-Patent Literature), patents, patent application publications, and patent applications mentioned in this specification are indicative of the level of skill of those skilled in the art to which this invention pertains. All such publications (e.g., Non-Patent Literature), patents, patent application publications, and patent applications are herein incorporated by reference to the same extent as if each individual publication, patent, patent application publication, or patent application was specifically and individually indicated to be incorporated by reference.

Although methods and materials similar or equivalent to those described herein may be used in the invention or testing of the present invention, suitable methods and materials are described herein. The materials, methods and examples are illustrative only, and are not intended to be limiting.

The invention now being generally described, it will be more readily understood by reference to the following examples, which are included merely for purposes of illustration of certain aspects and embodiments of the present invention, and are not intended to limit the invention.

### EXAMPLES

### Example 1

### Ultrasonic Cavitation Protocol for Isolation of Stromal Vascular Fraction

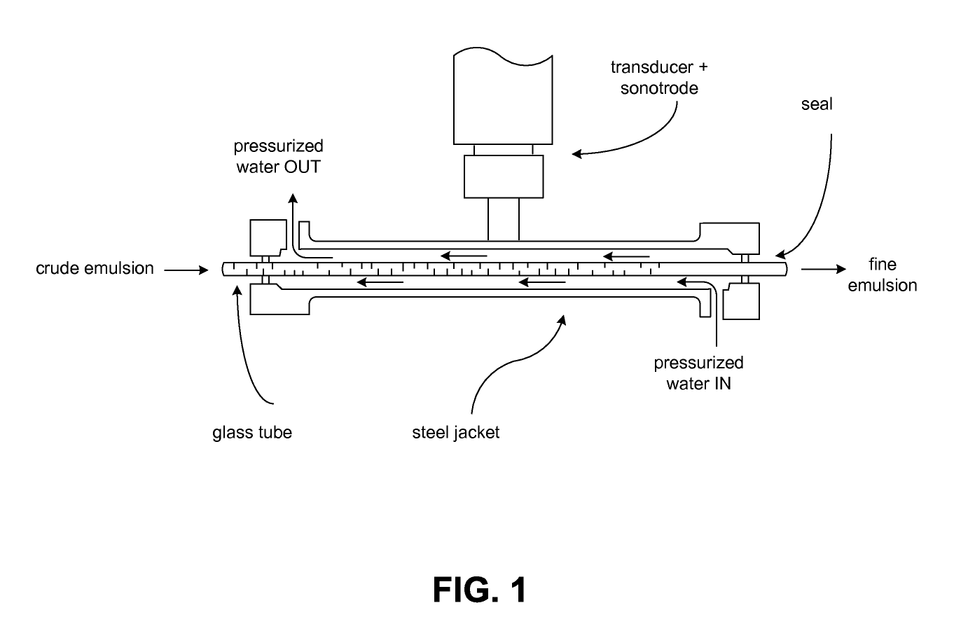

The following laboratory protocol was used to process adipose tissue and derive a stromal vascular fraction containing stem cells from adipose tissue (e.g., collected from patients as taught in the examples). It is to be understood that the protocol is exemplary and that the specifics may be modified by a skilled artisan in order to further optimize. Using this protocol, the inventor has processed hundreds of samples with consistently good results. As disclosed herein, and substantiated by Millipore studies (See FIGS. 1A-E, 2A-E, 3A-E, 4A-E and 5A-E) the subject ultrasonication protocol results in about 10-fold more viable cells than comparable adipose samples treated with collagenase. Also, the inventive methods result in the same cell population and cell types as collagenase isolation procedures, suggesting that the inventive methods preserve the integrity of all the desired stromal vascular fraction cells, and especially the cell types identified herein.

Turn on Laminar Flow hood 3 minutes prior to procedure. Set up Laminar Flow hood with sterile disposable drapes and tubes. Turn on Millipore Guava and check software. Check Gauge on Laminar Flow hood.

Attach the probe#14 (with a rode size of about 13-14 mm) to the Ultrasonic machine with a 200 W generator and tighten it with a wrench to be secured in place. Log in fat into Guava flow cytometer computer. Add about 50 mL of lipoaspirate (adipose tissue) to tube. A timer is used on 10 min. preset. Place probe into fat (make sure the probe does not touch the plastic). Slowly increase Cycle and Amplitude once the probe is submerged into syringe with fat, until reach Cycle 0.9 and Amplitude 90%. The ultrasonic cavitation was performed at 24 kHz. After 5 min. stop the ultrasonic process and raise the probe to a level of 40 cc on the syringe; check the sample and make sure that is not overflowing.

Remove specimen from Ultrasonic and pour the contents into a red top sterile conical specimen tube for filtering. Divide in equal amounts in two sterile red top conical specimen tubes, then add equal amounts of 0.9% Sodium Chloride. Centrifuge both specimens for 3 min. at 500 RCF (relative centrifugal force). When spinning is complete you will have a specimen that is layered, liquid on the bottom (with a pellet) and fat on top. Using a 20 cc syringe and metal infusion cannula attachment (spinal needles) submerge to bottom of specimen tube and remove liquid stem cells solution including the pellet (from this sample take approximately 2 cc of liquid to be used for testing with Flow Cytometer).

Pipette sample SVF into specimen tube. Pipette Guava reagent into sample and mix. Place sample into dark for 5-20 minutes. Place sample into flow cytometer. Run Guava Soft program. Record all results. Exemplary results are shown in Tables 2 and 3:

TABLE 2
Yields

| Experiment | Adipose Tissue | Cell Yield | Cell Viability |
|---|---|---|---|
| A Lipoaspirate | 60 mL (cc) | 12,800,000/cc | 86.5% |
| B Lipoaspirate | 60 mL (cc) | 5,930,000/cc | 94.3% |
| C Lipoaspirate | 60 mL (cc) | 3,930,000/cc | 96.2% |

TABLE 3
Cell Markers

| Experiment | CD14 | CD31 | CD45 | CD34 | CD29 | CD73 | CD90 | CD166 | CD105 | LIN1 | CD44 |
|---|---|---|---|---|---|---|---|---|---|---|---|
| A Lipoaspirate | 40% | 15% | 1% | 1% | 60% | 4% | 60% | 30% | 70% | 50% | 40% |
| B Lipoaspirate | 10% | 4% | 1% | 1% | 20% | 1% | 50% | 4% | 30% | 8% | 3% |
| C Lipoaspirate | 9% | 8% | 1% | 1% | 35% | 1% | 55% | 7% | 50% | 10% | 5% |

The results therein show that the subject ultrasonication protocol results in about 10-fold more viable cells than comparable adipose samples (same amount of adipose tissue) which were treated with an enzyme that breaks down collagen (collagenase). The results therein further show that the inventive methods result in the same cell population and cell types as collagenase isolation procedures, suggesting that the inventive methods preserve the integrity of all the desired stromal vascular fraction cells, and especially the cell types identified herein.

In contrast, U.S. Patent Application Publication No. 2006/0051865 describes the use of ultrasonic methods to release adult stem cells from adipose tissue. See Example 2. When the inventor reproduced their methods and disclosed operating conditions they were ineffective, i.e., they yielded few stromal vascular fraction cells. As measured by flow cytometry, the unprocessed lipoaspirate yields about 500,000 cells/ml, the 2006/0051865 protocol is a slight improvement at 700,000 cells/ml. In contrast, using the methods described herein, the inventor has been able to isolate 2,000,000 up to 22,000,000 cells/ml. Thus, the claimed method provides a yield of four times up to thirty times as much as the 2006/0051865 protocol. Thus, by contrast, the present invention consistently produces results in very high numbers of viable stromal vascular fraction cells, which are well suited for use in cell therapy or cosmetic procedures.

Thus, using the ultrasonic cavitation methods described herein one may isolate high levels of viable stromal vascular fraction cells from adipose tissue.

### Example 2

### Preparation of Adipose Tissue from Human Donor

### Method 1: Preparation of an Aspirate Containing Adipose Tissue by Liposuction

An excess amount of Tumescent solution (saline containing 0.0001% adrenalin), which exceeds the amount of liposuction to be aspirated prior to the liposuction operation, is infused into hypodermic fat layer (tumescent method), and thereafter cannulae having 2-3 mm of inner diameter (made of metal with aspirator) are used for the liposuction operation. Liposuction operations are well known in the art, and for example, can be referred to in Safe Liposuction and Fat Transfer Rhoda Narins (Ed) Marcel Dekker, Inc (2005) and Textbook of Liposuction Hanke, et al. Informa Plc. (2007).

Aspirated fat is washed with saline. About five to ten liters of washed aspirate was generated, and the resultant adipose tissue derived cellular materials are used for derivation of stromal vascular fractions.

### Method 2: Preparation of Adipose Fat Tissue by Surgery

Fat tissue was obtained by surgery from human subjects who had given their informed consent. Separation was conducted with techniques well known in the art. Briefly, human fat tissue was aseptically separated from fat tissue suctioned from human subjects who had given their informed consent. The resultant adipose tissue derived cellular materials are used for derivation of stromal vascular fractions.

Method 3: Harvesting Adipose Fat Tissue from Cadaver

Fat tissue may be obtained from a human cadaver using methods known in the art. The adipose tissue may be refrigerated (i.e., 4° C.), frozen (i.e., −20° C.), stored in liquid nitrogen (i.e., −70° C.).

### Example 3

### Preparation of a Stem Cell Suspension from an Aspirate of Liposuction

Adipose tissue derived from liposuction aspirates or surgically as described in the previous example are placed in a suitable tube and a biologic solution if desired (e.g., phosphate buffered saline solution or normal saline solution) and the adipose tissue in the composition is placed contact with the ultrasonic probe of an ultrasonic cavitation device as described in the Materials and Methods section above.

In particular, the Amplitude is set at about 50-100%, typically about 100%, Cycle 0.1-1.0 and about 50 cc fat lipoaspirate is placed into a tube, 60 cc tube size, 28 mm diameter and 110 mm length and is treated by ultrasonic cavitation for about 10 minutes where at 5 minutes, the ultracaviation is stopped and the probe is adjust upward towards the middle of the sample and continued for the remaining 5 minutes using a 14 mm ultrasonic rod.

The device may be set at about 50-100% intensity and frequency of about 10-100% for about 5-60 minutes for about 50 cc of adipose tissue. This treatment explodes the fat cells and thereby releases the stromal vascular fraction into the biologic solution, e.g., phosphate buffered or normal saline. As noted this treatment does not include the addition of collagenase or equivalent enzyme intended to break down collagen as cell dissociation is instead accomplished by ultrasonic sonication.

Preferably after ultrasonication the resultant solution is allowed to settle over time or is treated by centrifugation. The fat will float to the top. This solution will contain the stromal vascular fraction at the bottom which includes adipose-derived stem cells, endothelial cell precursors and other cells and this fraction is uncontaminated by exogenous enzymes such as collagenases.

The fat containing supernatant may be discarded. In addition as the desired cells may also float, an aspirator may be used to carefully perform suction without damaging the cells.

### Example 4

### Characterization of Recovered Stem Cells

The stromal vascular fraction containing stem cells recovered in Example 2 and using the Protocol above is characterized by known methods, e.g., flow cytometry or FACS, e.g., using antibodies that detect markers expressed on mesenchymal and stromal adipose derived stem cells. These methods will detect the presence of viable stem cells.

It is to be understood that the protocols disclosed herein are exemplary and that the specifics may be modified by a skilled artisan in order to further optimize. Using the specific protocol reported in the Example 1, the applicant has processed over 200 samples with consistently good results. The stem cells resulting therefrom have been used to treat patients. In addition, the applicant has compared the stem cell containing cell samples derived according to the invention to those derived by conventional procedures (collagenase derived samples). More specifically, adipose-derived stem cell samples produced according to the invention were compared to those obtained in a study by Millipore. The comparison revealed that the inventive ultrasonic cavitation procedures result in the same cell population. Unexpectedly, the inventive procedure is much more efficient, i.e., it consistently results in about 10 times the number of cells for the same amount of fat.

For example, the inventor compared three methods of isolating stromal vascular or mesenchymal vascular cells including lipoaspirate, the protocol of U.S. Patent Application Publication No. 2006/0051865, and the method described herein. As measured by flow cytometry, lipoaspirate yields about 500,000 cells/ml, the U.S. Patent Application Publication No. 2006/0051865 protocol is a slight improvement at 700,000 cells/ml. In contrast, using the method described herein, the inventor isolated 2,000,000 up to 22,000,000 cells/ml. This was an unexpected result because sonication is considered in the art for lysing cells and the length of ultracaviation (i.e., 10 minutes) was unusual as compared to what was tried in the art.

### Example 5

### Post-mortem Adipose Tissue from Animals

Chicken fat was purchased from the butcher. The chicken was killed the day before and kept on ice chips. The fat was exposed to ultrasonic cavitation protocol described in Example 1 and the Stromal Vascular Fraction counts were as follows: Viability: 86.5%, Cell Count: 1.28×10⁷ cells per mL, and Debris: 5.57%.

Beef fat was purchased from the butcher. It is estimated that it was aged for approximately 28 days at refrigerated temperatures (4° C.). The fat was exposed to ultrasonic cavitation protocol described in Example 1 and the Stromal Vascular Fraction counts were as follows: Viability: 96.6%, Cell Count: 3.49×10⁶ cells per mL, Debris: 1.28%.

Blend approximately 2 ounces of chicken fat to a uniform smooth consistency for approximately 60 seconds. Place 30 cc's of the fat into a 60 cc syringe. Place the syringe into the holding arm in a vertical position on the sonicator unit. The ultrasound cavitation rod is placed into the syringe at the 15 cc mark (or ½ the depth of the sample). Make sure the sonicator settings are set to the lowest settings and then turn on the unit. Slowly turn up the Amplitude setting to 90% then slowly turn up the Cycle setting to 0.9 Set the timer and let the unit run for 5 minutes. After 5 minutes is up, turn off the sonicator and turn the Amplitude and Cycle knobs back to the lowest settings.

Remove the 60 cc syringe from the holding arm and pour equal amounts of the sonicated lipo-aspirate tissue into two 50 cc conical tubes. Calculate the amount of specimen there is (usually 30 cc's) and add an equal amount of sterile 0.9% NaCl injectable solution to each of the two specimens in the 50 cc conical tubes. Filter each specimen by screwing on a Millipore steriflip 100 micro-meter filter and use a syringe to create suction to draw the liquid solution across the membrane. Once all of the liquid has been transferred from one conical tube to the other, unscrew the filter and put the cap back on the conical tube.

Transfer the SVF into four (4) 15 cc conical tubes. Put the conical tubes inside of the centrifuge and spin them for 6 minutes at 2800 RPM. The SVF cells to be used are contained in a pellet that drops to the bottom of the conical tube via gravity. The top of the conical contains the centrifuged fat and a white membrane like sub-structure that contains primarily white blood cells.

Use a 16 gauge blunt tip spinal needle attached to a 25 cc syringe to draw up the SVF cells that made a pellet on the bottom of the conical tubes. This will be approximately 20 cc. Aliquot approximately 1 cc of cells from each syringe into an Eppendorf® tube for flow cytometer testing.

TABLE 4

| Experiment | Adipose Tissue | Cell Yield | Cell Viability |
|---|---|---|---|
| 1 Chicken | 30 mL (cc) | 12,800,000/cc | 86.5% |
| 2 Chicken | 30 mL (cc) | 6,730,000/cc | 76% |
| 3 Beef | 30 mL (cc) | 3,490,000/cc | 96.6% |

Thus the ultrasonic cavitation method described herein may be used on post-mortem sources of adipose tissue to isolate a stromal vascular fraction with high cell yields and high cell viability.

### Example 6

### Homogenization with Beads

Stromal vascular fraction cells may be isolated from adipose tissue by means of homogenization with beads. A stored lipoaspirate sample from a human donor was defrosted and aliquoted into tube samples containing 7 mL of adipose tissue and 3 mL of tumescent fluid (a mixture of saline, Lidocaine®, and a vasoconstrictor, i.e., epinephrine).

One level scoop, approximately 5 mL of 2 mm zirconium oxide beads, was added to the sample. The tube was then placed into the Bullet Blender® Blue 50 instrument manufactured by Next Advance. The sample was homogenized on speed 1 for 3 minutes.

Approximately 10 mL of saline was then added to the sample. The sample was then filtered with a 100 μm steriflip filter (100 to 50 micron nylon material filters). The sample was then prepared for testing on the Millipore easyCyte flow cytometer, the results of which are described in Table 5.

TABLE 5
Cell Yield from Homogenization with Beads

| Sample Volume (mL) | Cell Count | Viability | Debris |
|---|---|---|---|
| 7 | 2.3 × 10⁶ | 88% | 3.68% |

Thus the homogenization with beads method described herein may be used on adipose tissue to isolate a stromal vascular fraction with high cell yields and high cell viability.

### Example 7

### In Situ Extraction and Isolation of Stromal Vascular Fraction

A patient is selected to undergo identification and extraction of tissue, optionally connective, muscle, adipose, nervous, or epithelial tissue. The tissue is identified using an ultrasound method. The tissue is thereby extracted by and subject to ultrasonic cavitation using an ultrasound flow cell. The stromal vascular fraction is then isolated.

### Example 8

### Indirect Sonication Protocol

Indirect Sonicator from Q Sonica was used to obtain SVF from lipoaspirate. 60 cc of lipoaspirate was placed in a metal bowl which was then submerged in water in order to facilitate the transfer of sound waves from the probe to the lipoaspirate. The sample was sonicated for about 30 minutes at an amplitude of about 100. The power was set to about 200 W. After the sonication, normal saline was added to the sample in a 50/50 ratio and the sample was then spun to harvest the pellet. The pellet was tested on flow cytometer. Some of the results include a cell count of 1.67×10⁷ and 2.24×10⁷. Viabilities were 98.9% and 97.1%

TABLE 6
Human Samples (adipose tissue)

| Human Sample | Cell Count/mL | Cell viability |
|---|---|---|
| 1 | 4.00 × 10⁶ | 91.20% |
| 2 | 1.39 × 10⁷ | 94.60% |
| 3 | 1.04 × 10⁷ | 97.20% |
| 4 | 1.02 × 10⁷ | 92.80% |
| 5 | 9.96 × 10⁶ | 80.38% |
| 6 | 1.71 × 10⁷ | 97.30% |
| 7 | 1.41 × 10⁷ | 97.10% |
| 8 | 1.79 × 10⁷ | 96.50% |
| 9 | 9.78 × 10⁶ | 98.60% |
| 10 | 2.20 × 10⁶ | 97.00% |
| AVERAGE | 1.09 × 10⁷ | 94.27% |

TABLE 7
Other tissue (nonliving)

| Tissue type (nonliving) | Cell count/mL | Viability (%) |
|---|---|---|
| Abdomen adipose | 1.26 × 10⁷ | 90.8 |
| Omental adipose | 9.1 × 10⁶ | 84.9 |
| Gluteal adipose | 3.09 × 10⁶ | 89.2 |
| Dog adipose | 5.51 × 10⁵ | 88.0 |
| Chicken adipose | 8.9 × 10⁶ | 87.5 |

Thus indirect cavitation may be used to isolate stromal vascular fraction from tissues, optionally adipose, different species, and different species with high cell yields and high cell viability.

Those skilled in the art will recognize, or be able to ascertain using no more than routine experimentation, many equivalents to the specific embodiments of the invention described herein. Such equivalents are intended to be encompassed by the following claims.

1. A method for isolating a stromal vascular fraction from a tissue comprising subjecting said tissue to mechanical, chemical, and/or enzymatic treatment to release the stromal vascular fraction.

2. The method of claim 1, wherein said method comprises subjecting said tissue to ultrasonic cavitation, wherein the cells in the tissue and blood vessels in the tissue are lysed, thereby dissociating or releasing substantial numbers of intact stromal vascular fraction cells from the lysed blood vessels contained in the ultrasonicated tissue while substantially maintaining the viability of the cells constituting the stromal vascular fraction.

3. The method of claim 1, wherein said method comprises subjecting a tissue to ultrasonic cavitation comprising bringing said tissue into contact with a powerful ultrasonic cup horn, ultrasonic probe (rod), ultrasonic flow cell, or ultrasonic microtiter plate.

4. The method of claim 3, wherein said method is performed in situ in a patient.

5. The method of claim 4, wherein said method further comprises removing the lysed tissue comprising the lysed blood vessels and the stromal vascular fraction, optionally adipose tissue.

6. The method of claim 2, wherein said method further comprises identifying and targeting the tissue using ultrasound.

7. The method of claim 2, wherein said ultrasonic cavitation is applied directly externally to the tissue, optionally applied to the patient's skin and the blood vessels are lysed to release the stromal vascular fraction.

8. The method of claim 2, wherein the ultrasonic cavitation rod is inserted through a puncture or incision in the skin into the patient's tissue and lyse the blood vessels to release stromal vascular fraction.

9. The method of claim 2, wherein the tissue is located using ultrasound device and then removed using indirect cavitation device and subject to ultrasonic cavitation to release the stromal vascular fraction.

10. The method of claim 2, wherein the tissue is, optionally located using ultrasound device, removed subject to ultrasonic cavitation by an ultrasonic flow cell device, optionally, an Ultrasonic Mini Flow Cell, to release the stromal vascular fraction, optionally, wherein the stromal vascular fraction is further isolated.

11-14. (canceled)

15. The method of claim 1, wherein said ultrasonic cavitation is performed using an ultrasonic probe of about 10-15 mm, about 13 or 14 mm probe, optionally about 14 mm.

16. The method of claim 15, wherein said probe is 14 mm.

17. (canceled)

18. The method of claim 1, wherein said method comprises providing a tissue obtained from an animal; mixing said a tissue with tumescent fluid; and homogenizing said tissue with beads for about 30 seconds to 6 minutes, wherein the tissue cells and blood vessels in the tissue are lysed, thereby dissociating or releasing substantial numbers of intact stromal vascular fraction cells from the lysed blood vessels contained in the tissue while substantially maintaining the viability of the cells constituting the stromal vascular fraction.

19. (canceled)

20. (canceled)

21. The method of claim 18, wherein said beads are stainless steel, zirconium oxide, tungsten carbide, ceramic, zirconium silicate, or glass beads, optionally zirconium oxide beads.

22-37. (canceled)

38. The method of claim 1, wherein said method further comprises isolating the stromal vascular fraction (SVF).

39. The method of claim 1, wherein said method does not include the addition of an endopeptidase, optionally collagenase.

40. (canceled)

41. (canceled)

42. The method of claim 1, wherein the tissue is obtained from the stromal or mesenchymal compartment of a human cadaver, tissue bank, organ donation, solid fat obtained from a human cadaver, or a liposuction derived aspirate.

43. (canceled)

44. (canceled)

45. (canceled)

46. The method of claim 1, wherein said tissue comprises blood vessels.

47-65. (canceled)

66. The method of claim 1, wherein said stromal vascular cells are essentially free of T cells.

67. (canceled)

68. A method for cosmetic surgery comprising administering a stromal vascular fraction obtained by the method of claim 1.

69. (canceled)

70. (canceled)

71. (canceled)

72. A method of treating a disease comprising administering a stromal vascular fraction obtained by the method of claim 1.

73. (canceled)

74. (canceled)

75. A method for treating a disease comprising administering an allogeneic transplant comprising stromal vascular cells obtained by the method of claim 1.

76-156. (canceled)
